(19) United States
(12) Patent Application Publication
(10) Pub. No.: US 2020/0063105 A1
(43) Pub. Date: Feb. 27, 2020

(54) TRANSCRIPTION FACTORS CONTROLLING DIFFERENTIATION OF STEM CELLS

(71) Applicant: President and Fellows of Harvard College, Cambridge, MA (US)

(72) Inventors: Hon Man Alex Ng, Cambridge, MA (US); George M. Church, Brookline, MA (US); Volker Busskamp, Dresden (DE)

(73) Assignee: President and Fellows of Harvard College, Cambridge, MA (US)

(21) Appl. No.: 16/609,982

(22) PCT Filed: Apr. 30, 2018
(86) PCT No.: PCT/US2018/030216

§ 371 Date: Oct. 31, 2019

Related U.S. Application Data
(60) Provisional application No. 62/517,307, filed on Jun. 9, 2017.
(60) Provisional application No. 62/492,552, filed on May 1, 2017.

(51) Int. Cl. C12N 5/077; C07K 14/47; A61K 35/35; G01N 33/50

(52) U.S. Cl. CPC C12N 5/0652; C07K 14/4702; C12N 2501/60; G01N 33/5005; C12N 2506/45; A61K 35/35

(57) ABSTRACT

Forced expression of a handful of transcription factors (TFs) can induce conversions between cell identities; however, the extent to which TFs can alter cell identity has not been systematically assessed. Here, we assembled a “human TFome,” a comprehensive expression library of 1,578 human TF clones with full coverage of the major TF families. By systematically screening the human TFome, we identified many individual TFs that induce loss of human-induced-pluripotent-stem-cell (hiPSC) identity, suggesting a pervasive ability for TFs to alter cell identity. Using large-scale computational cell type classification trained on thousands of tissue expression profiles, we identified cell types generated by these TFs with high efficiency and speed, without additional selections or mechanical perturbations. TF expression in adult human tissues only correlated with some of the cell lineage generated, suggesting more complexity than observation studies can explain.

### RELATED APPLICATION DATA

This application claims priority to U.S. Provisional Application No. 62/492,552 filed on May 1, 2017 and U.S. Provisional Application No. 62/517,307 filed on Jun. 9, 2017, each of which is hereby incorporated herein by reference in its entirety for all purposes.

### STATEMENT OF GOVERNMENT INTERESTS

This invention was made with government support under 5P50HG005550 and HC008525 awarded by the National Institutes of Health. The government has certain rights in the invention.

### TECHNICAL FIELD OF THE INVENTION

This invention is related to the area of cell fates. In particular, it relates to induction of differentiated cells on the one hand and to the maintenance of stem cells on the other hand.

### BACKGROUND OF THE INVENTION

The discovery of mouse embryonic stem cells (ESCs), human ESCs and mouse and human induced pluripotent stem cells (hiPSC) has expanded the working modes in biology: unlike studying biological phenomena such as differentiation, gaining and maintaining cellular identity in vivo, we are now theoretically able to mimic some of these processes in a dish. The use of hiPSCs facilitates studying the genesis of human cell types in an ethically approved setting, and enables production of medically relevant cell types for research and medicine. However, exploiting the full differentiation potency of stem cells is only possible with few differentiated cell types. So far, stem cell differentiation protocols are multifaceted and tailored to individual cell types. These protocols often yield highly heterogeneous populations, which may mask the cell type of interest. Whereas the initial triggers that drive stem cells out of pluripotency are known, we know very little about subsequent molecular events that occur during differentiation. For example, initial triggers include the application of differentiation media, the addition of small molecules, application of growth factors or 3D culturing techniques. These stimuli act via cellular signaling cascades and converge on transcription factors (TFs), which alter gene expression by activation and/or repression. Some of these cascades may recapitulate in vivo development, for example retinogenesis in 3D retinal organoids whereas monolayer cultures seem to follow alternative molecular differentiation routes although final cell types can have high similarity with in vivo cell types. Examples of cell identity transitions, both during the course of natural development in vivo and in synthetic in vitro systems, demonstrate the importance of controlling these transcriptional programs. The ability to control cell identity has led to the current preoccupation of the stem cell field, as facile access to primary-like human cells would enable many applications in disease modeling, drug screening and regenerative medicine. Fundamentally, the ability to find and control transitions between cell identities would greatly enhance our understanding of cell identity.

Upon obtaining a cellular identity, its maintenance is a central feature of multicellular organisms. This comprises terminally differentiated cells but also physiological stem cells, which can give rise to specific cell types in the body. To maintain the stem cell pool, transcriptional programs are active in large part by transcription factors that prevent these cells from differentiating. In vitro, maintaining the pluripotency of ESCs or iPSCs is essential to expand and maintain these cells for downstream applications. Labor-intensive and delicate culturing techniques have been developed to maintain pluripotency and to avoid spontaneous differentiation. Advances in the formulation of culture media have significantly improved the robustness of stem cell maintenance, made feeder cell co-cultures dispensable and opened the usage of stem cells to a larger scientific community.

Due to the dependence on transcriptional programs to maintain or convert cellular identities it would be desirable to gain direct control at the transcriptomic level. Detailed study of the transcriptional control of the lac operon was the first example that a certain class of DNA-binding proteins, transcription factors, initiates, enhances or represses transcription. It was shown that one could obtain transcriptional control on cell fate conversion within the same germ layer by ectopic TF expression, which was pioneered by overexpressing MyoD in fibroblasts, which subsequently converted to myoblasts, and C/EBP-alpha to convert B cells into macrophages. Forced TF induction can also convert cells arising from different germ layers, pioneered by the three TFs Brn2, Ascl1 and Mytl1 (BAM) that convert fibroblasts into neurons. It has also been shown that certain sets of TF can change the identity of neurons in living animals. Another striking example is the generation of iPSCs by overexpressing Oct3/4, Sox2, Klf4 and c-Myc (“Yamanaka factors”) in fibroblasts. TF choices were mostly “biologically inspired;” the ones known from in vivo development were tested in vitro manually or proposed by computational approaches. This biased selection of ectopic TFs led to the successful generation of a handful of cell types. Notably, the failure rate of selected TFs to induce desired cell types from stem cells is relatively high, either not working at all or resulting in unexpected cell fates. Due to experimental and technical differences, for example gene delivery routes, transient or inducible TF expression or differences in starting cell types, we cannot easily troubleshoot these results. Since the forced TF expression in human stem cells can be very efficient, we wondered how one could confer this differentiation route systematically on producing other cell types. Obviously, one needs to standardize the technical aspects of TF delivery, expression, screening and analysis. In contrast to “biologically-inspired” TFs, an unbiased and systematic TF library screen to identify novel TFs that convert stem cells to other cell types or which reinforce pluripotency would be desirable and complementary.

So far, there have not been unbiased, systematic open reading frame (ORF) screens for converting cell identity. More recently, CRISPR-based activator screens have been performed but not for cell conversion. CRISPR-based activators for cell conversion appear to be insufficient to overcome barriers for cell identity conversion. Systematic, RNAi-based screens have revealed important pluripotency factors to understand stem cell biology, but over-expression to maintain pluripotency has not been systematically performed.

Cell types derived from human induced pluripotent stem cells (hiPSC) have high relevance for biomedical research and medicine, but robust, efficient and rapid protocols are lacking for many cell type: could we generate new protocols to generate cell types?

There is a continuing need in the art for methods of changing cell fate and maintaining cell fate so that cell populations available for cellular transplantation and drug screening can better reflect the diverse cell types in the human body.

### SUMMARY OF THE INVENTION

One aspect of the invention is a method of inducing differentiation of induced pluripotent stem cells. A nucleic acid comprising an open reading frame (ORF) encoding a transcription factor, the transcription factor (TF) protein, or an activator of transcription of the gene encoding the transcription factor, is delivered to the induced pluripotent stem cells. As a consequence, the amount of the transcription factor in the induced pluripotent stem cells is increased, and the induced pluripotent stem cells differentiate to form differentiated cells. The transcription factor may be one or more of the group consisting of ASCL1; ASCL4; ATF1; ATF4; ATF7; ATOH1; ATTB1; ATXN7; BARHL2; BARX1; BATF3; BHLHA15; BOLA1; BOLA2; BOLA2B; BSX; CAMTA2; CDX1; CDX2; CEBPZ; CIZ1; CREB; CREB3; CREB3L1; CREB3L4; CREBL2; DACH2; DLX1; DLX3; DMRT1; DRGX; DUXA; E2F2; EBF3; ELP3; EMX1; EN1; EN2; EPAS1; ETV1; ETV2; FIGLA; FLI1; FLJ12895; FOXB1; FOXC1; FOXD1; FOXD2; FOXD4L2; FOXE1; FOXF1; FOXF2; FOXH1; FOXI2; FOXL1; FOXN2; FOXO6; FOXP1; FOXR1; FOXR2; GBX2; GCM2; GLI1; GLIS1; GLIS3; GRHL1; GRHL2; GRHL3; GRLF1; GSC2; GZF1; HAND2; HES2; HES3; HES7; HIC2; HLX; HMGA1; HMX2; HOXA1; HOXA10; HOXA6; HOXB13; HOXB7; HOXB8; HOXB9; HOXC10; HOXC5; HOXD3; HSF1; HSFY1; ID3; ID4; IKZF1; IKZF3; INSM2; IRF2; IRF3; IRF7; IRF9; ISL2; KDM2A; KDM4E; KIAA0961; KLF6; KLF8; LBX2; LEUTX; LHX5; LMX1A; LOC51058; LOC91661; LYL1; MAEL; MAFK; MBNL3; MEF2B; MEF2C; MEIS1; MEIS3; MITF; MKX; MSC; MSGN1; MSX1; MXD3; MXD4; NEUROD4; NEUROD6; NEUROG1; NEUROG2; NEUROG3; NFIC; NFX1; NFYB; NKX2-2; NKX2-6; NKX2-8; NKX3-1; NKX3-2; NKX6-1; NKX6-3; NOTO; NPAS4; NR1B2; NR1H3; NR1H4; NR1I3; NR2F2; NR3C1; NR3C2; NRF1; NRL; OSR2; OTEX; OTP; OVOL1; OVOL2; PAX5; PAX9; PDX1; PEPP-2; PITX2; PKNOX2; PLAG1; PLAGL1; POU2F3; POU5F1B; PRDM5; PRDM6; PRDM7; PRDM9; PROX2; PRRX1; RAX2; REL; RELA; RFX1; RFX8; RFXANK; RUNX1; SALL3; SEBOX; SIM2; SIX2; SIX3; SIX4; SKIL; SMAD6; SMYD2; SOX10; SOX12; SOX13; SOX14; SOX21; SOX3; SPIB; SREBF1; TBX10; TBX18; TBX3; TBX5; TBX6; TCF1; TCF12; TCF15; TCF2; TCF21; TCF7; TCF7L2; TERF1; TFAP2B; TFCP2L1; TFDP2; TFDP3; TFE3; TGIF1; TLX1; TLX2; TLX3; TSC22D3; TUB; UBP1; UNCX; UNKL; USF1; VSX2; WDHD1; XBP1; YY1; ZBTB1; ZBTB2; ZBTB24; ZBTB25; ZBTB41; ZBTB44; ZBTB7C; ZBTB8B; ZFP36L2; ZFP64; ZFP92; ZFPM1; ZFX; ZGLP1; ZIC3; ZMAT1; ZMAT3; ZNF124; ZNF14; ZNF143; ZNF148; ZNF157; ZNF16; ZNF169; ZNF177; ZNF182; ZNF19; ZNF197; ZNF226; ZNF230; ZNF235; ZNF239; ZNF254; ZNF26; ZNF271; ZNF273; ZNF3; ZNF305; ZNF316; ZNF319; ZNF320; ZNF324; ZNF324B; ZNF326; ZNF333; ZNF34; ZNF347; ZNF350; ZNF395; ZNF396; ZNF404; ZNF408; ZNF415; ZNF426; ZNF44; ZNF440; ZNF441; ZNF442; ZNF443; ZNF449; ZNF45; ZNF454; ZNF470; ZNF474; ZNF480; ZNF490; ZNF499; ZNF506; ZNF507; ZNF512; ZNF514; ZNF516; ZNF518B; ZNF519; ZNF524; ZNF543; ZNF547; ZNF548; ZNF556; ZNF560; ZNF576; ZNF582; ZNF584; ZNF585B; ZNF592; ZNF593; ZNF594; ZNF599; ZNF600; ZNF616; ZNF626; ZNF639; ZNF643; ZNF646; ZNF652; ZNF653; ZNF66; ZNF660; ZNF664; ZNF668; ZNF671; ZNF678; ZNF682; ZNF683; ZNF692; ZNF706; ZNF709; ZNF714; ZNF716; ZNF717; ZNF718; ZNF720; ZNF721; ZNF729; ZNF749; ZNF75A; ZNF776; ZNF777; ZNF780B; ZNF783; ZNF785; ZNF791; ZNF799; ZNF8; ZNF808; ZNF835; ZNF84; ZNF841; ZNF843; ZNF85; ZNF852; ZSCAN22; ZSCAN23; ZSCAN29; ZSCAN5A; ZSCAN5C; ZXDB; MITF; NKX3-2; ZNF643; CDX2; ZNF777; SOX14; ZNF3; MKX; ZNF474; NEUROG1; DMRT1; PRDM5; NEUROG3; ZNF273; FOXP1; MXD4; NRL; E2F2; ZNF44; ZNF616; SOX12; HOXA10; GLI1; LMX1A; TBX10; EPAS1; HOXB7; PRDM7; RELA; OVOL2; BARX1; ATOH1; ZNF169; TSC22D3; NEUROG2; FOXD2; FOXI2; NR1B2; MEF2C; CAMTA2; HOXC5; ZNF230; IRF2; TFDP3; RUNX1; MSC; ZNF320; EN1; ZNF84; RAX2; NR1H3; DLX3; ZNF148; LHX5; BOLA2; TGIF1; BOLA1; ATF7; ID3; HMGA1; ZNF783; MAFK; GRHL1; FLI1; HES7; MAEL; ETV2; ZSCAN5A; HOXA1; FOXF2; KLF6; MBNL3; HES2; ATTB1; GRLF1; ZNF593; XBP1; ZNF326; MSGN1; BATF3; ID4; ZIC3; ZNF718; MXD3; ZNF426. ZNF706; ZNF652; FOXD1; GBX2; NEUROD4; ZNF490; ZNF85; SOX3; ZNF653; SIM2; HOXC10; ZNF26; FOXB1; ZNF668; ZNF576; NKX6-1; CDX1; MEF2B; DLX1; ZNF776; ZNF16; EN2; HOXA6; NKX2-8; CREBL2; DRGX; DUXA; ZNF396; ZNF692; VSX2; NR3C2; NR1I3; FIGLA; TLX1; HMX2; ASCL4; ZNF324B; ZNF75A; TCF21; BHLHA15; NKX6-3; ZNF720; TCF15; PITX2; HLX; ZNF124; SIX2; IKZF3; ZNF626; RFXANK; ATF4; ZNF678; ZNF514; GRHL2; FOXR2; ZNF660; CREB1; TFAP2B; ZMAT3; TCF7L2; FOXR1; OVOL1; HOXB13; HAND2; TCF12; UNKL; ZNF324; ZNF333; ZNF843; CREB3; ZBTB24; TERF1; ZNF785; FOXE1; OSR2; ZNF45; FOXL1; TBX3; ZNF157; NKX2-2; ZNF671; PAX9; NFRC; TBX6; NKX3-1; ZNF319; ZNF408; ZNF584; PKNOX2; SOX13; ZFP36L2; FLJ12895; NFX1; NR3C1; ZNF395; GUIS1; GLIS3; ZNF560; ZNF683; PLAGL1; SKIL; TCF1; UBP1; KLF8; ZNF239; TBX5; FOXF1; ZNF664; PRRX1; ISL2; FOXD4L2; ZNF143; KDM4E; ELP3; ZNF440; PEPP-2; PLAG1; TFE3; ZNF226; NEUROD6; TCF2; SMYD2; ZNF499; ZNF639; ZNF480; ZNF182; KDM2A; NPAS4; LOC51058; ATF1; SPIB; TLX2; ZBTB1; POU2F3; ZSCAN23; TUB; ZNF547; SIX4; KIAA0961; TFCP2L1; TCF7; ZNF177; YY1; ZNF585B; ZNF271; ZNF305; ZBTB2; OTP; NFYB; OTEX; ZNF443; ZNF852; ZNF646; ZNF835; ZNF682; ZNF66; ZNF235; SIX3; MEIS1; ZFPM1; ZNF441; LYL1; HOXB9; GSC2; ZBTB7C; ZNF518B; FOXH1; ZNF516; ZNF594; ZNF716; ZNF714; ZNF780B; ZNF470; CIZ1; RFX1; ZNF592; SOX21; LBX2; ZNF709; MSX1; LOC91661; PROX2; ZNF316; ZNF717; ZNF197; POU5F1B; ZBTB44; ZSCAN22; ZNF791; CREB3L1; GRHL3; REL; SOX10; SALL3; HSFY1; ZNF350; ZNF8; ZMAT1; NRF1; ZNF582; TBX18; ZFX; ZNF404; ZNF449; ZNF721; PDX1; SREBF1; GCM2; ZNF454; ZNF507; NR1H4; LEUTX; ZNF841; ZNF14; HES3; ASCL1; ZGLP1; INSM2; EMX1; HSF1; TFDP2; ZNF548; ZFP92; CREB3L4; CEBPZ; IKZF1; ZSCAN5C; FOXN2; ZNF524; ZFP64; EBF3; ZNF34; ZNF254; ZNF512; ZNF729; ZNF600; BOLA2B; BSX; WDHD1; ZNF19; ZNF543; PRDM9; ZXDB; ZBTB25; GZF1; NOTO; HOXD3; ZBTB41; ZNF442; IRF7; DACH2; IRF3; RFX8; NR2F2; ZBTB8B; PRDM6; ZNF808; ZNF556; ATXN7; ZSCAN29; NKX2-6; ZNF599; HOXB8; ZNF347; HIC2; BARHL2; ZNF506; FOXC1; ZNF415; PAX5; FOXO6; ZNF749; ZNF799; TLX3; UNCX; ETV1; SEBOX; MEIS3; IRF9; ZNF519; USF1; and SMAD6.

Another aspect of the invention is a method of maintaining pluripotency of induced pluripotent stem cells. A nucleic acid comprising an open reading frame encoding the protein, the protein, or an activator of transcription of an open reading frame encoding the protein is delivered to the induced pluripotent stem cells. The protein is a transcription factor that is found in a high proportion of stem cells relative to differentiated cells after delivery of a library of transcription factors to a population of induced pluripotent stem cells. As a consequence of the delivery, the induced pluripotent stem cells maintain expression of keratin sulfate cell surface antigen, TRA-1-60, which is a marker of stem cell identity.

Another aspect of the invention is an engineered human differentiated cell that comprises one or more nucleic acids comprising an open reading frame. The one or more ORFs encodes a transcription factor selected from the group consisting of ATOH1, NEUROG3, {NEUROG1 and NEUROG2}, {NEUROG1 and NEUROG2 and EMX1}, {NEUROG1 and NEUROG2 and EMX2}, {NEUROG1 and NEUROG2 and TBR1}, {NEUROG1 and NEUROG2 and FOXG1}, ETV2, MYOG, FOXC1, MITF, SOX14, WT1, TFPD3, CDX2, SMAD3, and ZSCAN1.

Yet another aspect of the invention is an engineered human differentiated cell which comprises a nucleic acid comprising an open reading frame encoding a protein. The protein is selected from the group consisting of ZNF70; ZNF461; TFAP2B; ZNF426; MITF; CDX2; MEOX2; AKNA; NKX2-8; NKX3-2; NKX2-3; ZNF16/HZF1; ETV2; TFDP3; RELA/p65; NEUROG1; ID4; HES7; MXD4; SOX14; FOXP1; E2F2; NEUROG3; ZNF148/ZBP89; GRLF1/ARHGAP35; BOLA2; ZNF616; MAX; ATOH1; PRDM5; LHX5; ZNF273; MAFK; HOXA1; HIF2A/EPAS1; MAFB; E2F3; PRDM7; ZNF44; HMGA1; NRL; BATF3; MYOG; KLF15; LMX1A; HOXB6; DMRTB1; ATF7; SCRT2; ZNF593; HES2; ZSCAN2; MSX2; ID3; SOX12; GLI1; DPRX; SMAD3; ZBED3; CAMTA2; MSC; ASCL3; BARX1; DMRT1; HOXA10; TSC22D3; ZNF837; MXD3; ZNF692; NHLH2; ZNF626; THAP3; SRY; WT1; SHOX; ZNF43; and ZSCAN1.

Still another aspect of the invention is an induced pluripotent stem cell which comprises a nucleic acid comprising an open reading frame encoding a protein. The protein is a transcription factor that is found in a high proportion of stem cells relative to differentiated cells after delivery of a library of transcription factors to a population of induced pluripotent stem cells. The induced pluripotent stem cell expresses keratin sulfate cell surface antigen. TRA-1-60. The transcription factor may be one or more of the group consisting of AEBP2; AIRE; ARNT2; ARNTL; ATF6; BACH1; BARX2; CASZ1; CLOCK; CREBRF; CTCFL; CXXC1; DLX2; E2F1; EBF1; EGR1; ELF4; ELF5; EMX2; ETS1; ETV1; ETV7; FEZF1; FOXJ3; FOXL2; FOXN2; FOXO1; FOXP3; FOXS1; GATA1; GCM1; HMBOX1; HOXB9; HOXC10; HOXC6; HOXD10; HOXD3; HOXD8; HSF2; HSFY1; IRF5; IRF9; IRX2; KCMF1; KLF1; KLF13; KLF16; KLF7; KLF8; LBX1; LCORL; MAF; MEF2A; MEIS2; MNT; MYBL2; MYF6; MYRF; NANOG; NEUROD1; NFE2L2; NFE2L3; NFIB; NFIX; NKRF; NOTO; NROB1; NR1B1; NR1B3; NR1C2; NR1C3; NR1F3; NR1I2; NR2B2; NR2C1; NR3A1; NR3A2; NR3C4; NR5A2; OSR1; PAX6; PAX7; PAX9; PHOX2B; PKNOX1; PLAGL1; PREB; PRRX1; RBPJ; RC3H2; RFX5; SATB1; SMAD2; SMAD4; SP1; SP4; SP6; SREBF1; STAT3; TAL2; TBX14; TBX18; TBX21; TBX22; TBX5; TEAD2; TERF; TERF2; TFAP2A; TFDP2; TFEB; THAP1; THAP10; TP63; TSHZ3; YEATS2; ZBED2; ZBTB12; ZBTB14; ZBTB17; ZBTB21; ZBTB35; ZBTB9; ZEB1; ZFHX3; ZFPM2; ZFY; ZFYVE26; ZIC4; ZKSCAN1; ZKSCAN12; ZKSCAN14; ZKSCAN19; ZKSCAN24; ZKSCAN4; ZKSCAN5; ZMAT2; ZNF114; ZNF138; ZNF146; ZNF227; ZNF253; ZNF266; ZNF280A; ZNF280C; ZNF282; ZNF296; ZNF311; ZNF317; ZNF337; ZNF34; ZNF35; ZNF350; ZNF366; ZNF396; ZNF398; ZNF41; ZNF415; ZNF460; ZNF485; ZNF497; ZNF511; ZNF512; ZNF517; ZNF543; ZNF550; ZNF586; ZNF613; ZNF615; ZNF619; ZNF641; ZNF644; ZNF645; ZNF648; ZNF655; ZNF658; ZNF662; ZNF664; ZNF669; ZNF704; ZNF740; ZNIF754; ZNIF75A; ZNF774; ZNF789; ZNF8; ZNF80; ZNF84; ZSCAN16; ZSCAN22; ZSCAN32; ZXDA; ZXDC; and ZZZ3.

Another aspect of the invention is a method of inducing differentiation of induced pluripotent stem cells. Increased expression is induced of a gene selected from the group consisting of ATOH1, NEUROG3, (NEUROG1 and NEUROG2), (NEUROG1 and NEUROG2 and EMX1), (NEUROG1 and NEUROG2 and EMX2), (NEUROG1 and NEUROG2 and TBR1). (NEUROG1 and NEUROG2 and FOXG1), ETV2, MYOG, FOXC1, MITF, SOX14, WT1, TFPD3, CDX2, SMAD3, and ZSCAN1. The induced pluripotent stem cells thereby differentiate to form differentiated cells.

Yet another aspect of the invention is a method of inducing differentiation of induced pluripotent stem cells. Expression of a gene is induced. The gene is selected from the group consisting of ATOH1, NEUROG3, {NEUROG1 and NEUROG2}, {NEUROG1 and NEUROG2 and EMX1}, {NEUROG1 and NEUROG2 and EMX2}, {NEUROG1 and NEUROG2 and TBR1}, {NEUROG1 and NEUROG2 and FOXG1}, ETV2, MYOG, FOXC1, MITF, SOX14, WT1, TFPD3, CDX2, SMAD3, and ZSCAN1. The induced pluripotent stem cells thereby differentiate to form differentiated cells.

Yet another aspect of the invention is an engineered human differentiated cell which comprises a nucleic acid comprising an open reading frame encoding a protein. The protein is NKX3-2. The open reading frame may be intronless.

Another aspect of the invention is a method of inducing differentiation of induced pluripotent stem cells. Increased expression is induced of the NKX3-2 gene. The induced pluripotent stem cells thereby differentiate to form differentiated cells.

Yet another aspect of the invention is a method of inducing differentiation of induced pluripotent stem cells. Expression of the NKX3-2 gene is induced. The induced pluripotent stem cells thereby differentiate to form differentiated cells.

These and other embodiments which will be apparent to those of skill in the art upon reading the specification provide the art with tools and methods for controlling the cell fate of stem cell populations.

### BRIEF DESCRIPTION OF THE DRAWINGS

FIG. 1. Workflow for screening the human TFome (a comprehensive expression library of 1.578 human transcription factor (TF) clones with full coverage of the major TF families) for loss of pluripotency in hiPSCs (human-induced-pluripotent-stem-cell)

FIG. 2. General strategy for high-throughput screening for cell type conversion

FIG. 3. The Human TFome expression library

FIG. 4. Expression vectors for delivery of human TFome

FIG. 5. Hits from high-throughput screening for stem cell differentiation [loss of stem cell identity (TRA-1-60) in human induced pluripotent stem cells]

FIG. 6. Hits are enriched for developmental genes and protein domains

FIG. 7. Selected transcription factors that induce stem cell differentiation

FIG. 8. ATOH1 induces neuronal differentiation from hiPSCs in stem cell media without embryoid body formation, additional growth factors or mechanical manipulations

FIG. 9. NEUROG3 induces neuronal differentiation from hiPSCs in stem cell media without embryoid body formation, additional growth factors or mechanical manipulations

FIG. 10. ETV2 induces endothelial differentiation from hiPSCs in stem cell media without embryoid body formation, additional growth factors or mechanical manipulations

FIG. 11. MYOG induces muscle differentiation from hiPSCs in stem cell media without embryoid body formation, additional growth factors or mechanical manipulations

FIG. 12. FOXC1 induces differentiation of hiPSCs in stem cell media without embryoid body formation, additional growth factors or mechanical manipulations

FIG. 13. MITF induces differentiation of hiPSCs in stem cell media without embryoid body formation, additional growth factors or mechanical manipulations

FIG. 14. SOX14 induces hiPSC differentiation in stem cell media without embryoid body formation, additional growth factors or mechanical manipulations

FIG. 15. ZSCAN1 induces hiPSC differentiation in stem cell media without embryoid body formation, additional growth factors or mechanical manipulations

FIG. 16. WT1 induces hiPSC differentiation in stem cell media without embryoid body formation, additional growth factors or mechanical manipulations

FIG. 17. NEUROGENIN1 and NEUROGENIN2 induce neuronal differentiation from hiPSCs in stem cell media without embryoid body formation, additional growth factors or mechanical manipulations

FIG. 18. NEUROGENIN1 and NEUROGENIN2 and EMX1 induce cortical neuronal differentiation from hiPSCs in stem cell media without embryoid body formation, additional growth factors or mechanical manipulations

FIG. 19. NEUROGENIN1 and NEUROGENIN2 and EMX2 induce cortical neuronal differentiation from hiPSCs in stem cell media without embryoid body formation, additional growth factors or mechanical manipulations

FIG. 20. NEUROGENIN1 and NEUROGENIN2 and TBR1 induce cortical neuronal differentiation from hiPSCs in stem cell media without embryoid body formation, additional growth factors or mechanical manipulations

FIG. 21. NEUROGENIN1 and NEUROGENIN2 and FOXG1 induce cortical neuronal differentiation from hiPSCs in stem cell media without embryoid body formation, additional growth factors or mechanical manipulations

FIG. 22. Selected transcription factors (first nine shown) are expressed at a low level or not at all, compared to housekeeping genes (last three shown) in hiPSCs

FIG. 23. Average copy number of TF ORFs integrated into the hiPSC genome

FIG. 24A-24F. Examples of validation and characterization of TFome transcription factors (TFs). (FIG. 24A) Validation of TFs identified by TFome loss-of-pluripotency (LOP) screens as driving differentiation of hiPSC. Whereas the initial screen assesses LOP by loss of TRA 1-60 staining, validation screens look directly at NANOG, SOX2, and OCT4. Each pair of bars represents the loss of gene expression in hiPSC when the indicated TFome TF is induced vs. cells in which it is uninduced. (FIG. 24B) Clustering of TFome TFs identified as having high LOP by RNA-seq expression profiles, as represented by the first three Principal Components (PC). The clustering indicates that diverse cell types are generated, but only some clusters can be clearly associated with differentiated cell types. (FIG. 24C-FIG. 24D) TF FoxC1 drives differentiation towards a cardiac muscle fate, as shown by gene ontology enrichments scores (FIG. 24C) and expression of a subset of cardiac marker genes (FIG. 24D). When induced. TFome TFs HoxB6 and NKX32 also upregulate some cardiac markers. (FIG. 24E-FIG. 24F) Different TFome isoforms of the TF ETV2 differentiate hiPSC towards endothelial cells with different efficiencies. All four isoforms of ETV2 on the Uniprot database (indicated by their Uniprot accession numbers; see (FIG. 24E)) were expressed from TFome constructs in hiPSC. (FIG. 24F) Isoform 1 (000321-1) was highly potent and resulted in 90% of cells expressing endothelial marker VE-Cadherin, while isoform-2 (000321-2), which differs from isoform 1 only by having 28 additional amino acids, only induced VE-Cadherin in ˜50% cells. Isoforms K7ERX2 and Q3KNT2 only induced ˜20% and 0%, respectively. All experiments summarized in this Figure were conducted with PGP1 hiPSC containing piggyBac-integrated dox-inducible TFome constructs, for which dox was induced for 4 days in pluripotency-reinforcing media.

FIG. 25. Cells were pulsed with transcription factor ETV2 by induction of expression of the transcription factor for varying numbers of days as indicated. VE-cadherin, an endothelial marker, shows as purple and nuclei show as blue.

FIG. 26A-26D. NKX3-2 induces deterministic stromal cell differentiation. FIG. 26A: The percentage of VIM-positive cells was determined by flow cytometry. FIG. 26B: Cells were differentiated for four days, and RNA was harvested from differentiated cells and stem cells, and then sequenced and quantified FIG. 26C: Additional stromal cell markers were evaluated compared to stem cells on a log 2(stromal cell/stem cell) scale. FIG. 26D: Protein levels were determined by antibody staining. A single cell is magnified in the insert.

FIG. 27A-27B. TFome screen for loss of stem cell identity. (FIG. 27A) 367 TFs were statistically significant at inducing stem cell differentiation compared to uninduced control. (Combined from whole library screen with n=3 independent transductions, Wald test, and sub-pool screen with n=6, t-test). (FIG. 27B) Differentiation upon doxycycline induction after four days as assessed, validated by loss of any of NANOG, SOX2, or OCT4. Error bars show standard error of the mean (s.e.m.), (n=3, t-test). FIG. 28A-28B. Characterization of NKX3-2-induced stromal cells. (FIG. 27A) Brightfield images of NKX3-2 cells incubated with or without doxycycline for 4 days, then embedded in a collagen gel. Scale bar, 4 mm. (FIG. 27B) Quantification of cell surface area (n=3, t-test).

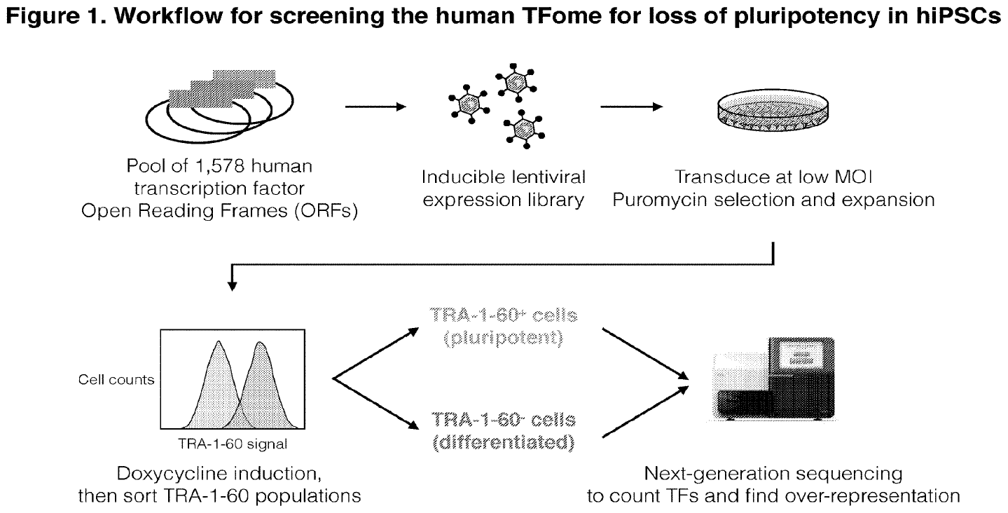

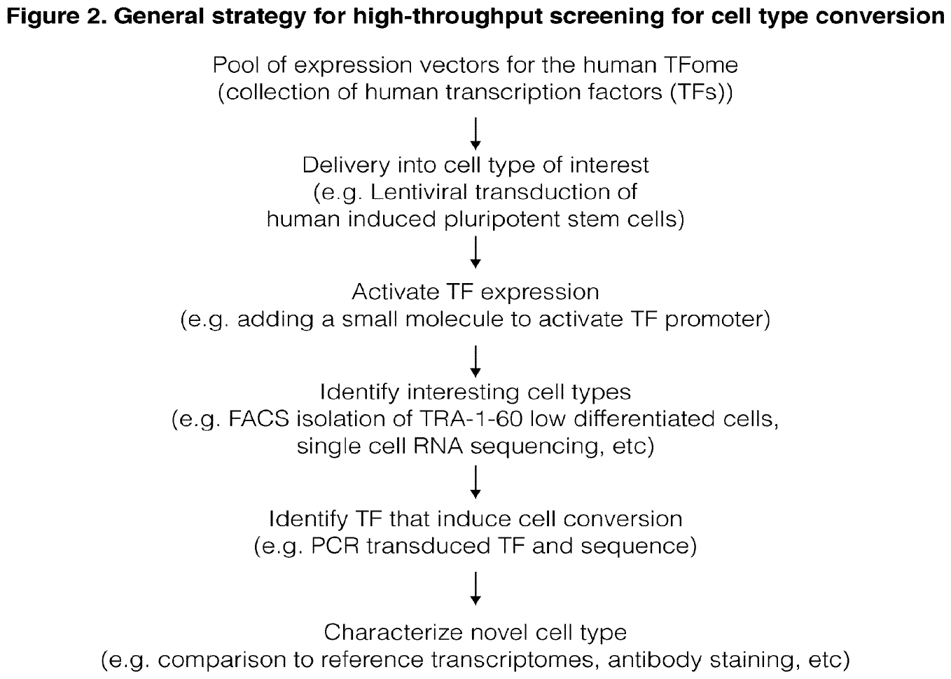

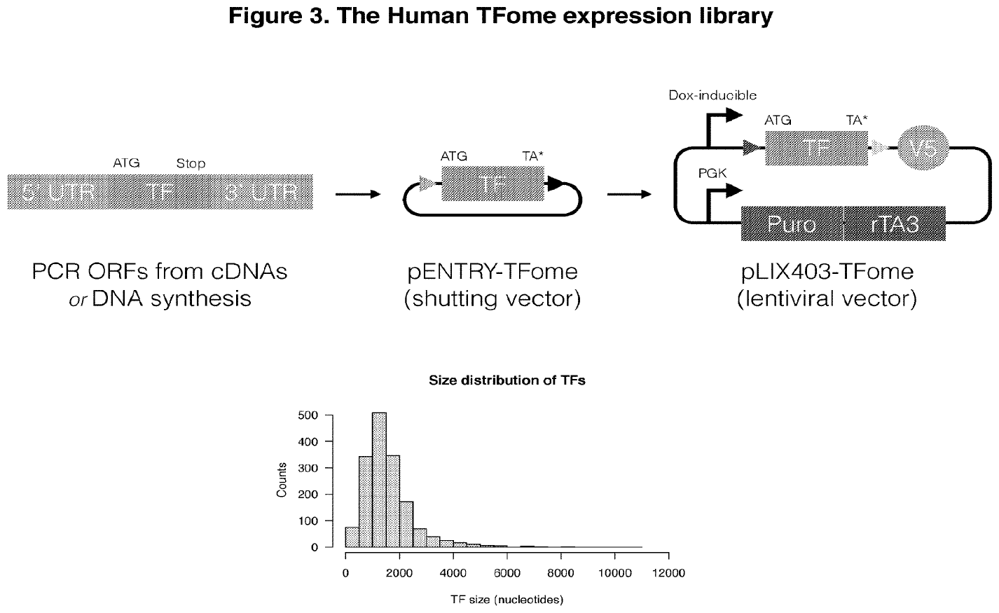

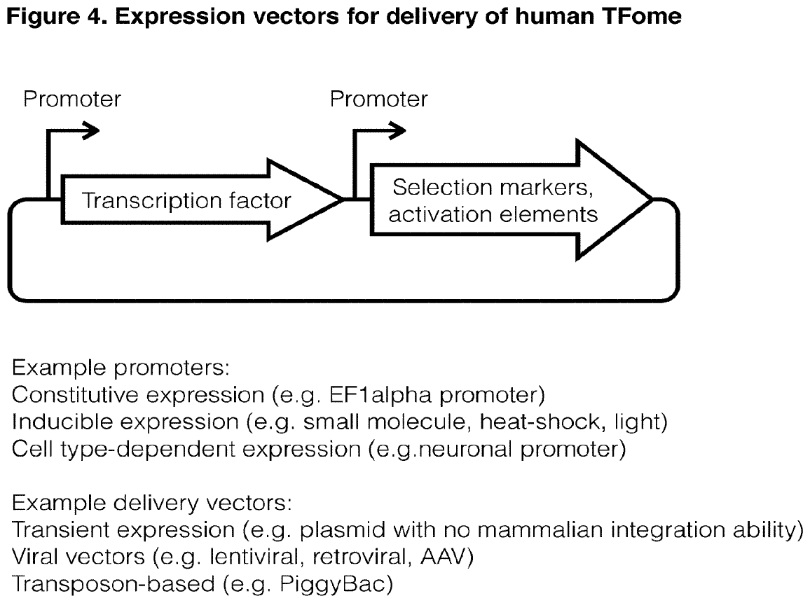

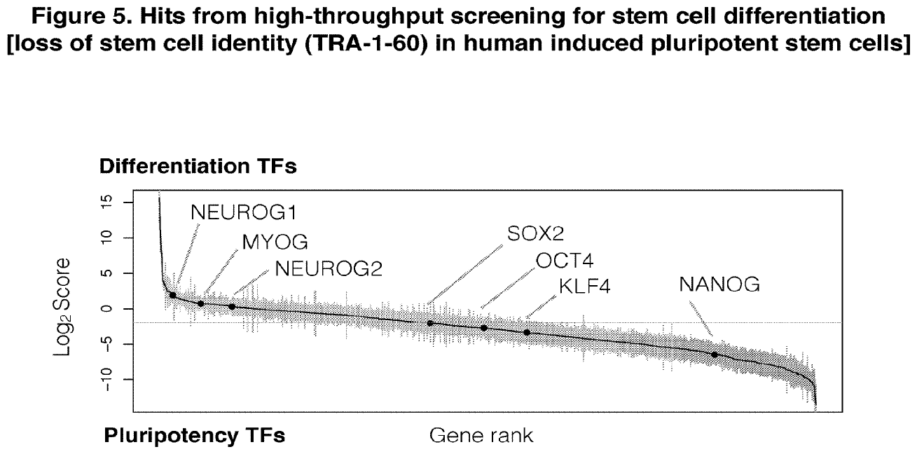

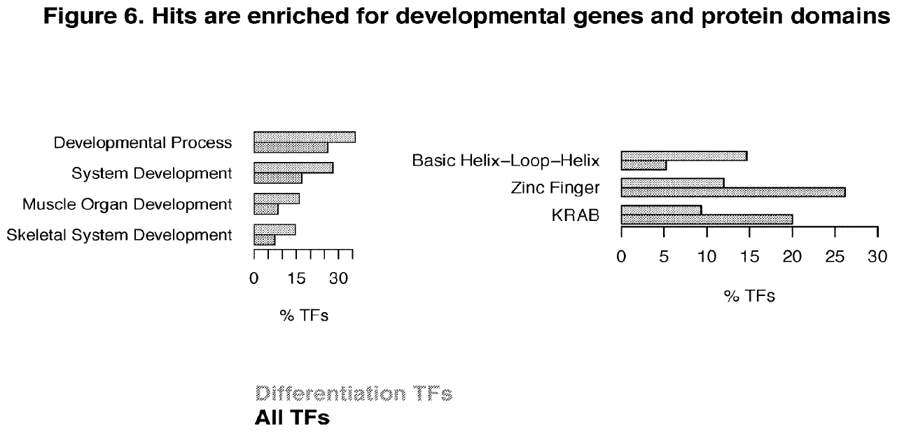

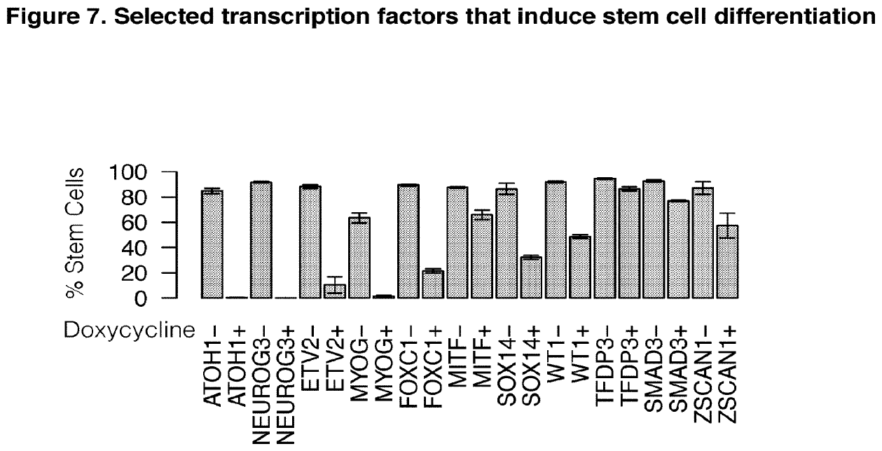

### DETAILED DESCRIPTION OF THE INVENTION

The inventors have developed techniques for inducing differentiation of stem cells into particular cell lineages, as well as techniques for inducing stem cells to continue to proliferate as stem cells. Rather than using special growth conditions, small molecules, or growth factors, the techniques deploy transcription factors (TFs) to trigger differentiation programs or stem cell renewal programs that prevent differentiation.

As determined, certain transcription factors are able to induce stem cells to differentiate to particular lineages. For example, MITF causes stem cells to form melanocytes. Similarly, CDX2 causes stem cells to form placental cells. MYOG causes stem cells to form smooth muscle cells. An initial indicator of differentiation can be the loss of stem cell specific markers. For example, the keratin sulfate cell surface antigen, TRA-1-60, can be assayed in the cells, as it is lost early in the process of differentiation. Using loss of stem cell markers as an indicator also provides a general means for detecting relevant transcription factors, rather than looking for acquisition of a marker that is newly expressed during differentiation by a particular cell lineage. Loss-of-marker screening may be applied to cell types other than stem cells, for instance, to identify TFs that directly convert fibroblasts into cell types of interest. In some aspects, combination of transcription factors may be used to achieve differentiation to a particular cell lineage. The combination may achieve a cell type or a cell sub-type that is not achieved by either transcription factor alone. Alternatively, the combination may achieve the same cell type as one of the transcription factors alone, but may achieve it more efficiently.

Stem cells may be sorted from differentiated cells or differentiating cells by various means, typically based on differential gene expression. For example, fluorescence activated cell sorting may be used to separate cells on the basis for their expression of TRA-1-60. Alternatively, certain differentiated cells may be sorted from other differentiated cells and from cells on the basis of their expression of a lineage-specific cell surface antigen. Yet another means is by assessing expression at the RNA level, by single cell RNA sequencing without any sorting or pre-selection step. Such techniques are known in the art and may be used as is suitable and convenient for a particular application.

Any means known in the art for increasing the amount of a transcription factor in a stem cell can be used. This may involve delivery of either a nucleic acid comprising an open reading frame encoding the transcription factor, delivery of the transcription factor itself, or delivery of an activator of the transcription factor or its expression. Any technique known in the art for such delivery may be used. For example, for delivery of a cDNA, a viral or plasmid vector may be used. The open reading frame (encoding any isoform of the TF) may be inducible or repressible for control, to achieve a suitable level of expression. The nucleic acid comprising the open reading frame may be a cDNA, an mRNA, or a synthetic or engineered nucleic acid. Some transcription factors may require a critical amount of expression to effectively induce differentiation, such as the equivalent of at least 5, 10, 15, 20, 25, or 50 copies of the ORF per cell. Any amount over a diploid number per cell is termed high copy number. Other factors may require less than a certain threshold of expression due to possible toxicity at high levels, such as less than 20, 10, or 5 copies per cell. Increased levels of expression may also be achieved by increasing the copy number of an ORF, for example, by using a higher copy number vector or by using a transposon. In some embodiments, nuclease-null or “dead” Cas9 variants may be used to activate the transcription of a desired transcription factor. See. e.g., Chavez et al., Nature Methods 13:563-569, 2016. In other embodiments, modified RNAs—RNAs that encode the transcription factor, but use synthetic nucleotides that improve stability and reduce degradation—may be used. In some cases, use of culture media adapted for a particular cell type may increase the expression of the ORF that induces expression of that cell type. Expression of an ORF can be increased from a non-expressed gene, from a gene expressed at a low level, or from a gene expressed at a robust level. Overexpression is expression at level that is higher than the level that is expressed before induction from a gene that is expressed at a low, medium or high level. A period of induction of less than 10, 5, 3, or 1 day may be sufficient to induce differentiation. Indeed, a period of less than 24, 18, 12, 6, 4, 2, or 1 hour may be sufficient to induce differentiation. A shorter period of induction may be sufficient particularly when an induced ORF is present in high copy number.

An exogenous open reading frame is typically an open reading frame that differs from the similar gene or mRNA in the cell. It may be engineered to have a different control sequence or sequences, such as promoter, operator, enhancer, terminator, etc. It may be engineered to have no introns. It may be engineered to be fused to a second open reading frame to which it is not fused in the human genome.

The differentiated cells that can be produced using the methods described here will have multiple applications. They can be used for regenerative medicine, such as transplanting the cells into a recipient in need of a certain type of cell. They can be used for drug testing, both in cell culture as well as after transplantation. The cells may be used to deliver a product to a part of a body, for example, if they naturally produce or are engineered to produce and secrete the product.

Drug testing in the cells may use substances that are known or unknown to have a certain biological activity. The substances may be elements, compounds or mixtures, whether natural or synthetic. The cells may be used to determine a desirable activity of a potential drug or conversely to determine undesirable effects of a substance or lack of such effects. The contacting of the substance with the cells may be in culture or in a human or animal body. The activity or side effects of the substance may be determined in vitro or in vivo, irrespective of where the contacting occurred.

Changes that are observed in the cells being tested are not limited. The cells can be observed for effects on cell growth, apoptosis, secreted products, expression of particular products, etc. The genome of these cells may be edited to match mutations found in patients with disease. Any type of assay known in the art for such changes may be used, including but not limited to immunological assays, morphological observations, histochemical stains, reverse transcription polymerase chain reaction, protein blots, mass spectrometry, hybridization assays, electrophysiology, etc.

The stem cells may be obtained from any source. One particularly useful source is human induced pluripotent stem cells. Mouse induced pluripotent stem cells and mouse embryonic stem cells may also be used, as well as such cells from other animals. The use of human embryonic stem cells may be regulated or ethically undesirable, but these may be used as well.

Differentiated cells may be identified by any property or set of properties that is characteristic or defining of that type of differentiated cell. For example, different cell types have a unique transcriptome. The transcriptome may be used as a means of matching and identifying an unknown cell type to a known cell type. The transcriptome may be used qualitatively or quantitatively. Similarly a proteome may be used a means of identifying an unknown differentiated cell type. Some cell types may be identifiable based on morphology, growth habit, secretion products, enzymatic activity, cellular function, and the like. Any means known in the art for identifying cells may be used.

The above disclosure generally describes the present invention. All references disclosed herein are expressly incorporated by reference. A more complete understanding can be obtained by reference to the following specific examples, which are provided herein for purposes of illustration only, and are not intended to limit the scope of the invention.

### Example 1

We decided to compose a complete TF ORF library by combining factors from existing resources. Absent TFs were obtained by de novo gene synthesis. This “human TFome”, comprises 1,578 canonical human TFs driven by an inducible promoter system within an all-in-one Tet-ON lentiviral vector backbone for stable integration in hiPSCs. By screening the human TFome we found over 70 TFs that induce loss of hiPSC identity, suggesting pervasive potency for TFs to alter cell identity. This resource, the entire library as well as individual TFs, will be publicly available at Addgene (non-profit plasmid repository) and a subset of hiPSC lines in which certain TFs can be induced will be available. We applied the human TFome as single TFs per hiPSCs to identify individual TFs that convert stem cells to other cell identities or reinforce pluripotency. High-throughput fluorescence cell sorting (FACS) in combination with next generation sequencing and subsequent bioinformatic analyses was performed to screen for TFs in two hiPSC lines. We show that expression levels are critical and can be elevated by using transposon-mediated integration of expression cassettes. The application of the human TFome in hiPSCs resulted in the validation of over ten TFs cell fate converting TFs. Furthermore, we discovered over one hundred TFs that reinforce pluripotency, which may help to improve the robustness of stem cell cultures.

### Example 2

Generating and Applying the Human TFome

Systematic and comprehensive TF-wide induction screening in human stem cells requires the availability of a TF-expression library. Notably, only partial human libraries, for example TFs included in the ORFeome, were accessible. Therefore, we decided to assemble TFs from available resources or by de novo gene synthesis to compile the “human TFome”, an expression library of 1,578 TFs representing all canonical human transcription factors (Vaquerizas et al. 2009) and further curated.

The pLIX403 lentiviral vector was chosen because it allows for genomic transgene integration, doxycycline-inducible TF expression from a Tet-On system and puromycin selection of transduced cells. The individual TFs in shuttling vectors (pENTR) were cloned into pLIX403 by pooled gateway cloning. To discriminate the ectopic TFs from intrinsic expression, the TFs were marked by a V5 epitope tag translated on the backbone downstream of the Gateway recombination site on the C-terminus of the TF. The pLIX403 vector comprises a second ubiquitous promoter cassette driving the rTA3 gene needed to activate the TetOn promoter in the presence of the small molecule doxycycline and bicistronically a puromycin selection marker. About 98% of the TFs were detectable by next-generation sequencing after subcloning in the DNA plasmid library, which subsequently was used to produce lentiviral particles.

We transduced the PGP1 from the Personal Genome Project and ATCC DYSO100 human iPSC lines with the human TFome at a low multiplicity of infection (MOI=0.1), such that cells would receive a single lentiviral integration at most. In total, we transfected both cell lines each with a complete human TFome pool as well as with two subpools that we have six independent transductions. To obtain sufficient coverage of the library, we ensured that on average each TF was present in at least one hundred cells after lentiviral transduction. Genes that confer resistance to the antibiotics bleomycin, blasticidin, and hygromycin, which should not induce differentiation, were also transduced as negative controls. Transduced cells were then selected for TF-integration using puromycin, expanded and doxycycline was added for four days for continuous TF induction. FACS separated the stained population into the differentiated TRA-1-60^{low} and the pluripotent TRA-1-60^{high} population. For each of these populations, the integrated genes were amplified using universal primer PCR, and sequenced.

### Example 3

Transcription Factors-Wide Screening for Alterations in Pluripotency in Human Induced Pluripotent Stem Cells

To identify TFs that induce stem cells into any differentiated cell, we devised a strategy to screen for individual TFs that potently cause loss of pluripotency rather than enriching for cells with any specific cell type marker. We used a fluorescence-activated cell sorting (FACS) approach, which enables multiplexed assessment of the human TFome followed by TF identification by using next-generation sequencing. To measure the loss of pluripotency, we stained for the keratin sulfate cell surface antigen TRA-1-60, which is rapidly lost upon exit from pluripotency. Importantly, because we aimed to identify potent inducers of differentiation, we performed the screen in the standard mTeSR1 stem cell media.

We first aimed to comprehensively identify single TFs that would induce loss of pluripotency. To score each TF for its differentiation potential, we computed a ratio of each TF by dividing the number of normalized reads sequenced in the differentiated TRA-1-60^{low} population versus the pluripotent TRA-1-60^{high} population. We expect a TF that induces differentiation to be highly enriched in the TRA-1-60^{low} population compared to the pluripotent TRA-1-60^{high} population, and hence have a high ratio. Conversely a TF that reduces spontaneous differentiation, for instance by maintaining pluripotency, would have fewer reads in the differentiated population compared to the pluripotent population. We set a threshold for a TF having no effect based on the score of the truncated fluorescent proteins. TFs that increase cell proliferation without affecting pluripotency would be expected to have differentiation scores similar to these truncated fluorescent proteins.

Based on this differentiation scoring metric, we identified over 70 TFs that induce loss of pluripotency (“differentiation-inducing TFs”) and over 100 TFs that reinforce pluripotency (“pluripotency-reinforcing TFs”), as compared to the truncated fluorescent protein controls. To assess the quality of this screen, we conducted gene set enrichment analysis (GSEA) on the differentiation-inducing TFs. We discovered that differentiation-inducing TFs are enriched for developmental processes and system development, which is consistent with their ability to induce loss of pluripotency in our screen. In terms of protein domains, basic helix-loop-helix DNA-binding domains, which are often present in TFs involved in development were enriched, whereas, zinc fingers and Küppel-associated box (KRAB) domains were depleted. Overall, these results suggest that our screen in human stem cells recovers developmentally important TFs that have been identified in model organisms in vivo.

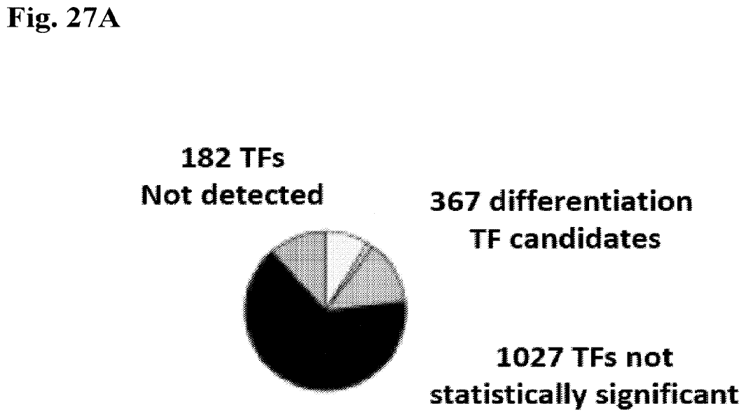

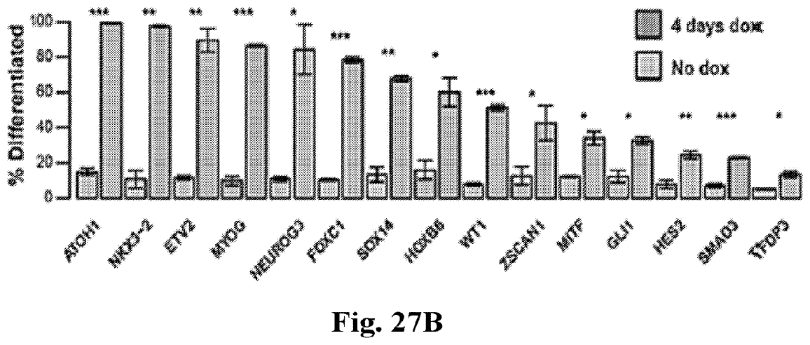

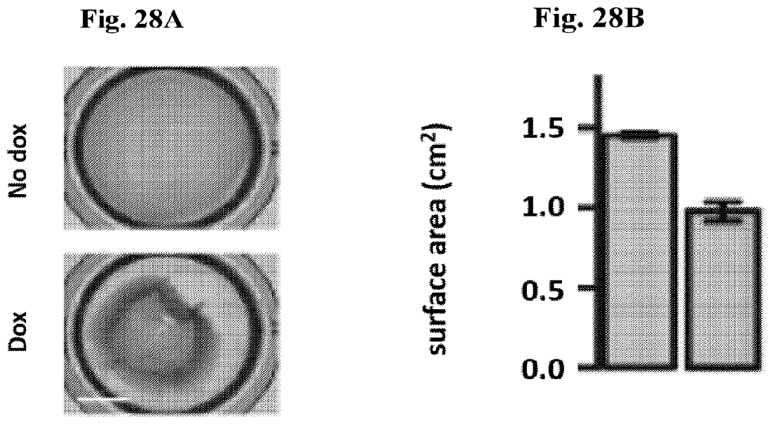

The genomic DNA of differentiated and stem cell samples were extracted, and ectopic TFs were identified by deep sequencing. For each TF, a differentiation score was computed based on the log 2 ratio of reads in the differentiated gate compared to the stem cell gate. We identified 160 differentiation-inducing candidate TFs, including known factors such as NEUROG2, which had statistically significant differentiation scores compared to non-induced control cells (FIG. 27A). When we repeated the screen we found additional transcription factors that induced differentiation. These are ASCL1; ASCL4; ATF1; ATF4; ATF7; ATOH1; ATTB1; ATXN7; BARHL2; BARX1; BATF3; BHLHA15; BOLA1; BOLA2; BOLA2B; BSX; CAMTA2; CDX1; CDX2; CEBPZ; CIZ1; CREB1; CREB3; CREB3L1; CREB3L4; CREBL2; DACH2; DLX1; DLX3; DMRT1; DRGX; DUXA; E2F2; EBF3; ELP3; EMX1; EN1; EN2; EPAS1; ETV1; ETV2; FIGLA; FLI1; FLJ12895; FOXB1; FOXC1; FOXD1; FOXD2; FOXD4L2; FOXE1; FOXF1; FOXF2; FOXH1; FOXI2; FOXL1; FOXN2; FOXO6; FOXP1; FOXR1; FOXR2; GBX2; GCM2; GLI1; GLIS1; GLIS3; GRHL1; GRHL2; GRHL3; GRLF1; GSC2; GZF1; HAND2; HES2; HES3; HES7; HIC2; HLX; HMGA1; HMX2; HOXA1; HOXA10; HOXA6; HOXB13; HOXB7; HOXB8; HOXB9; HOXC10; HOXC5; HOXD3; HSF1; HSFY1; ID3; ID4; IKZF1; IKZF3; INSM2; IRF2; IRF3; IRF7; IRF9; ISL2; KDM2A; KDM4E; KIAA0961; KLF6; KLF8; LBX2; LEUTX; LHX5; LMX1A; LOC51058; LOC91661; LYL1; MAEL; MAFK; MBNL3; MEF2B; MEF2C; MEIS1; MEIS3; MITF; MKX; MSC; MSGN1; MSX1; MXD3; MXD4; NEUROD4; NEUROD6; NEUROG1; NEUROG2; NEUROG3; NFIC; NFX1; NFYB; NKX2-2; NKX2-6; NKX2-8; NKX3-1; NKX3-2; NKX6-1; NKX6-3; NOTO; NPAS4; NR1B2; NR1H3; NR1H4; NR1I3; NR2F2; NR3C1; NR3C2; NRF1; NRL; OSR2; OTEX; OTP; OVOL1; OVOL2; PAX5; PAX9; PDX1; PEPP-2; PITX2; PKNOX2; PLAG1; PLAGL1; POU2F3; POU5F1B; PRDM5; PRDM6; PRDM7; PRDM9; PROX2; PRRX1; RAX2; REL; RELA; RFX1; RFX8; RFXANK; RUNX1; SALL3; SEBOX; SIM2; SIX2; SIX3; SIX4; SKIL; SMAD6; SMYD2; SOX10; SOX12; SOX13; SOX14; SOX21; SOX3; SPIB; SREBF1; TBX10; TBX18; TBX3; TBX5; TBX6; TCF1; TCF12; TCF15; TCF2; TCF21; TCF7; TCF7L2; TERF1; TFAP2B; TFCP2L1; TFDP2; TFDP3; TFE3; TGIF1; TLX1; TLX2; TLX3; TSC22D3; TUB; UBP1; UNCX; UNKL; USF1; VSX2; WDHD1; XBP1; YY1; ZBTB1; ZBTB2; ZBTB24; ZBTB25; ZBTB41; ZBTB44; ZBTB7C; ZBTB8B; ZFP36L2; ZFP64; ZFP92; ZFPM1; ZFX; ZGLP1; ZIC3; ZMAT1; ZMAT3; ZNF124; ZNF14; ZNF143; ZNF148; ZNF157; ZNF16; ZNF169; ZNF177; ZNF182; ZNF19; ZNF197; ZNF226; ZNF230; ZNF235; ZNF239; ZNF254; ZNF26; ZNF271; ZNF273; ZNF3; ZNF305; ZNF316; ZNF319; ZNF320; ZNF324; ZNF324B; ZNF326; ZNF333; ZNF34; ZNF347; ZNF350; ZNF395; ZNF396; ZNF404; ZNF408; ZNF415; ZNF426; ZNF44; ZNF440; ZNF441; ZNF442; ZNF443; ZNF449; ZNF4S; ZNF454; ZNF470; ZNF474; ZNF480; ZNF490; ZNF499; ZNF506; ZNF507; ZNF512; ZNF514; ZNF516; ZNF518B; ZNF519; ZNF524; ZNF543; ZNF547; ZNF548; ZNF556; ZNF560; ZNF576; ZNF582; ZNF584; ZNF585B; ZNF592; ZNF593; ZNF594; ZNF599; ZNF600; ZNF616; ZNF626; ZNF639; ZNF643; ZNF646; ZNF652; ZNF653; ZNF66; ZNF660; ZNF664; ZNF668; ZNF671; ZNF678; ZNF682; ZNF683; ZNF692; ZNF706; ZNF709; ZNF714; ZNF716; ZNF717; ZNF718; ZNF720; ZNF721; ZNF729; ZNF749; ZNF75A; ZNF776; ZNF777; ZNF780B; ZNF783; ZNF785; ZNF791; ZNF799; ZNF8; ZNF808; ZNF835; ZNF84; ZNF841; ZNF843; ZNF85; ZNF852; ZSCAN22; ZSCAN23; ZSCAN29; ZSCAN5A; ZSCAN5C; ZXDB; MITF; NKX3-2; ZNF643; CDX2; ZNF777; SOX14; ZNF3; MKX; ZNF474; NEUROG1; DMRT1; PRDM5; NEUROG3; ZNF273; FOXP1; MXD4; NRL; E2F2; ZNF44; ZNF616; SOX12; HOXA10; GLI1; LMX1A; TBX10; EPAS1; HOXB7; PRDM7; RELA; OVOL2; BARX1; ATOH1; ZNF169; TSC22D3; NEUROG2; FOXD2; FOXI2; NR1B2; MEF2C; CAMTA2; HOXC5; ZNF230; IRF2; TFDP3; RUNX1; MSC; ZNF320; EN1; ZNF84; RAX2; NR1H3; DLX3; ZNF148; LHX5; BOLA2; TGIF1; BOLA1; ATF7; ID3; HMGA1; ZNF783; MAFK; GRHL1; FLI1; HES7; MAEL; ETV2; ZSCAN5A; HOXA1; FOXF2; KLF6; MBNL3; HES2; ATTB1; GRLF1; ZNF593; XBP1; ZNF326; MSGN1; BATF3; ID4; ZIC3; ZNF718; MXD3; ZNF426; ZNF706; ZNF652; FOXD1; GBX2; NEUROD4; ZNF490; ZNF85; SOX3; ZNF653; SIM2; HOXC10; ZNF26; FOXB1; ZNF668; ZNF576; NKX6-1; CDX1; MEF2B; DLX1; ZNF776; ZNF16; EN2; HOXA6; NKX2-8; CREBL2; DRGX; DUXA; ZNF396; ZNF692; VSX2; NR3C2; NR1I3; FIGLA; TLX1; HMX2; ASCL4; ZNF324B; ZNF75A; TCF21; BHLHA15; NKX6-3; ZNF720; TCF15; PITX2; HLX; ZNF124; SIX2; IKZF3; ZNF626; RFXANK; ATF4; ZNF678; ZNF514; GRHL2; FOXR2; ZNF660; CREB1; TFAP2B; ZMAT3; TCF7L2; FOXR1; OVOL1; HOXB13; HAND2; TCF12; UNKL; ZNF324; ZNF333; ZNF843; CREB3; ZBTB24; TERF1; ZNF785; FOXE1; OSR2; ZNF45; FOXL1; TBX3; ZNF157; NKX2-2; ZNF671; PAX9; NFRC; TBX6; NKX3-1; ZNF319; ZNF408; ZNF584; PKNOX2; SOX13; ZFP36L2; FLI12895; NFX1; NR3C1; ZNF395; GLIS1; GLIS3; ZNF560; ZNF683; PLAGL1; SKIL; TCF1; UBP1; KLF8; ZNF239; TBX5; FOXF1; ZNF664; PRRX1; ISL2; FOXD4L2; ZNF143; KDM4E; ELP3; ZNF440; PEPP-2; PLAG1; TFE3; ZNF226; NEUROD6; TCF2; SMYD2; ZNF499; ZNF639; ZNF480; ZNF182; KDM2A; NPAS4; LOC51058; ATF1; SPIB; TLX2; ZBTB1; POU2F3; ZSCAN23; TUB; ZNF547; SIX4; KIAA0961; TFCP2L1; TCF7; ZNF177; YY1; ZNF585B; ZNF271; ZNF305; ZBTB2; OTP; NFYB; OTEX; ZNF443; ZNF852; ZNF646; ZNF835; ZNF682; ZNF66; ZNF235; SIX3; MEIS1; ZFPM1; ZNF441; LYL1; HOXB9; GSC2; ZBTB7C; ZNF518B; FOXH1; ZNF516; ZNF594; ZNF716; ZNF714; ZNF780B; ZNF470; CIZ1; RFX1; ZNF592; SOX21; LBX2; ZNF709; MSX1; LOC91661; PROX2; ZNF316; ZNF717; ZNF197; POU5F1B; ZBTB44; ZSCAN22; ZNF791; CREB3L1; GRHL3; REL; SOX10; SALL3; HSFY1; ZNF350; ZNF8; ZMAT1; NRF1; ZNF582; TBX18; ZFX; ZNF404; ZNF449; ZNF721; PDX1; SREBF1; GCM2; ZNF454; ZNF507; NR1H4; LEUTX; ZNF841; ZNF14; HES3; ASCL1; ZGLP1; INSM2; EMX1; HSF1; TFDP2; ZNF548; ZFP92; CREB3L4; CEBPZ; IKZF1; ZSCAN5C; FOXN2; ZNF524; ZFP64; EBF3; ZNF34; ZNF254; ZNF512; ZNF729; ZNF600; BOLA2B; BSX; WDHD1; ZNF19; ZNF543; PRDM9; ZXDB; ZBTB25; GZF1; NOTO; HOXD3; ZBTB41; ZNF442; IRF7; DACH2; IRF3; RFX8; NR2F2; ZBTB8B; PRDM6; ZNF808; ZNF556; ATXN7; ZSCAN29; NKX2-6; ZNF599; HOXB8; ZNF347; HIC2; BARHL2; ZNF506; FOXC1; ZNF415; PAX5; FOXO6; ZNF749; ZNF799; TLX3; UNCX; ETV1; SEBOX; MEIS3; IRF9; ZNF519; USF1; and SMAD6.

We validated TF candidates based on their score and gene ontology. To streamline cell-line engineering, we employed a PiggyBac transposon vector, which could be directly electroporated to make inducible, stable stem cell lines for each candidate TF. We noticed that PiggyBac-integrated TF cell lines differentiated with higher efficiency than those generated by lentiviruses (88±2% and 57±2% differentiation, respectively). A set of 15 TFs were tested: they showed significant loss of pluripotency using the orthogonal markers NANOG, OCT4/POU5F1, and SOX2 (FIG. 27B) and altered morphology.

### Example 4

Transcription Factors Induce Rapid and Efficient Differentiation when Expressed at High Levels

To craft genetic recipes that rapidly and efficiently induce hiPSCs into cell types, we engineered inducible single-TF expressing cell lines that could produce cell types of interest on demand. We originally aimed to mimic closely the conditions of the screen, namely single-copy lentiviral integration. Our initial efforts successfully validated the screen, however the differentiation efficiencies were weak (˜10%), similar to CRISPR-Cas9 activator-based differentiation experiments. To overcome this challenge while keeping with our goal for simple differentiation protocols without additional growth factors, mechanical steps or selections, we wondered whether low TF expression levels of the integrated lentiviral vector was the bottleneck of highly efficient differentiation. We surmised that certain crucial target genes may be occluded with a low probability of TF binding and activation of transcription; thus a higher expression would in theory increase the probability of a successful binding and hence transcriptional event that may activate positive feedback loops that induce exit from pluripotency.

To express high levels of TF, we first tested transduction with high titer lentiviral particles, which resulted in massive cell death. To improve the transduction efficiency and the vialibility of hiPSCs, we constructed a PiggyBac transposon-based vector with similar doxycycline-inducible TF expression and the ability to use puromycin to select for transduced cells. Critically, this transposon system allows for facile high-copy integration of TFs; in our hands, we average ˜15 integrated copies per genome, as assessed by digital droplet PCR. Due to this integration efficiency, it is challenging to set copy numbers to a single TF per cell. Therefore, we decided not to repeat the screen with the lentiviral system but selected TFs that had a high differentiation score for subcloning into the PiggyBac vectors.

Using this high expression system, we assessed differentiation efficiency. By brightfield microscopy, we observed a rapid loss of stem cell morphology, specifically migration away from colonies and adoption of distinct cell morphologies. We quantified loss of stem cell identity by intracellular flow cytometry for NANOG, OCT4 and SOX2. Uninduced stem cells had >90% NANOG⁺ OCT4⁺ SOX2⁺ cells, whereas doxycycline-induced cells had <10% NANOG⁺ OCT4⁺ SOX2⁺ cells.

### Example 5

Systematic Classification of TF-Induced Lineages

Next, we needed an approach of identifying a priori the cell lineage being generated—we needed a method analogous to BLAST, but for tissue profiles. Several studies have been published that generally compare gene expression profiles to infer common drug targets, disease mutation effects, etc; however, the broad range of genes did not allow for rigorous and unambiguous identification of cell lineage against expression profiles catalogued in the Gene Expression Omnibus. A BLAST-like approach also requires an extensive reference panel to compare to; however the mechanistic gene regulatory network-based algorithm CellNet requires many samples per tissue, which is not currently available. We systematically use thousands of RNA-seq datasets from many tissues as training data. We adapted KeyGenes, a machine-learning-based algorithm, to systematically BLAST our transcriptomes against high-quality tissue expression datasets.

The TFs that induce loss of stem cell identity may be caused by conversion into differentiated cell types, or general loss of cell identity without a specific identity. To systematically determine what lineage these TF may be inducing, we first needed a systematic approach to classify cell types. We curated a set of large-scale human tissue expression profile studies to train a machine learning classifier for cell types. This curation comprises RNA-seq samples representing tissues from the GTEx study, Human Protein Expression Atlas and Illumina Human Body Map. We then applied this as training data for KeyGenes, LASSO regression-based classifier for cell type identification.

To determine what cell lineages are being generated by these TFs, we performed RNA-seq on each cell population and used them as query for the expanded KeyGenes classifier.

Interestingly, expression of these TFs in adult human tissue did not predict which lineages were produced in hiPSCs. For instance, ETV2 induces endothelial differentiation but is most highly expressed in the testis, ATOH1 activated a neuronal cell identity but is highly expressed in the colon and small intestine, and CDX2 induced a placental identity, but is also highly expressed in the colon and small intestine. Thus observational studies alone do not appear to predict which TFs can induce which lineages, and suggest that synthetic methods of converting cell type do not necessarily recapitulate in vivo development.

Expression of specific markers for these cell lineages was present from the RNA-seq data). To enhance differentiation into these lineages, we induced TF expression in the presence of standard culturing conditions for those lineages, and assess conversion efficiency for lineage-specific markers by flow cytometry. Morphologies and lineage-specific marker expression were tested by immunohistochemical staining. Together, these data indicate that TF-derived cells can be potently and rapidly generated.

### Example 6

Controlling hiPSC Differentiation Via Human Transcription Factor Overexpression

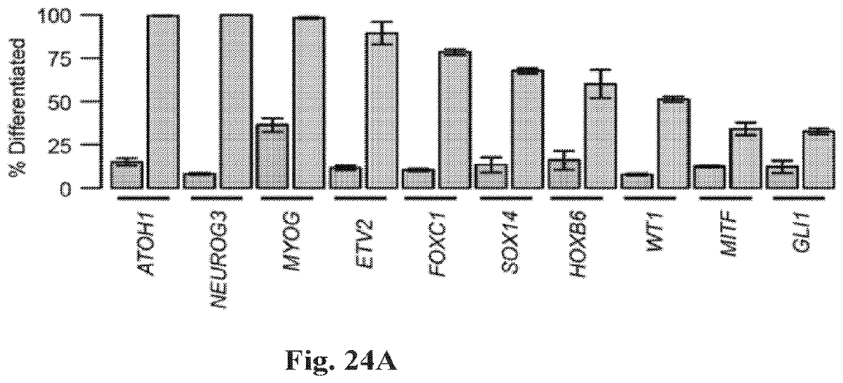

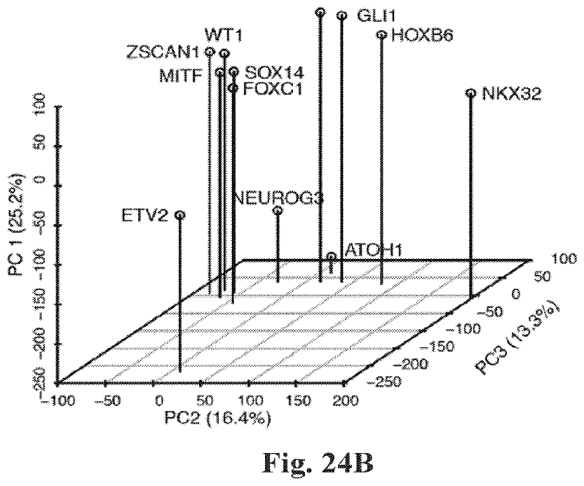

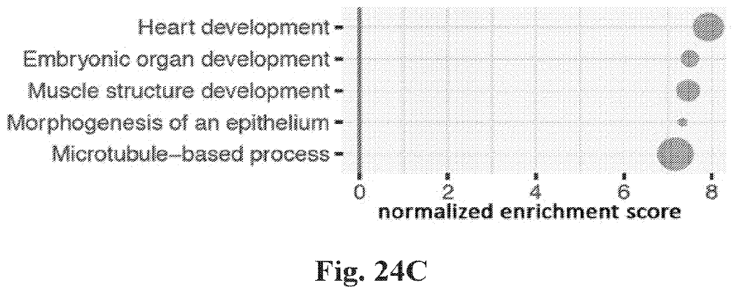

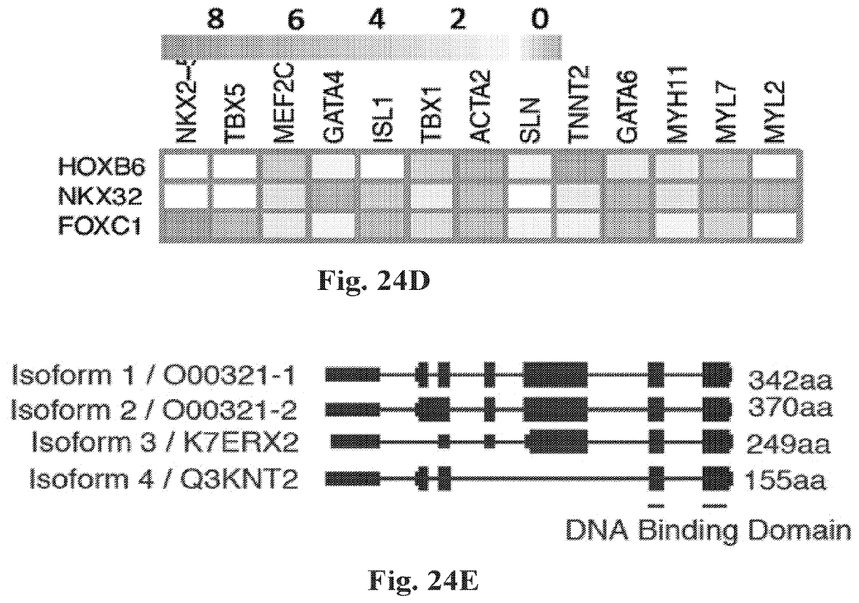

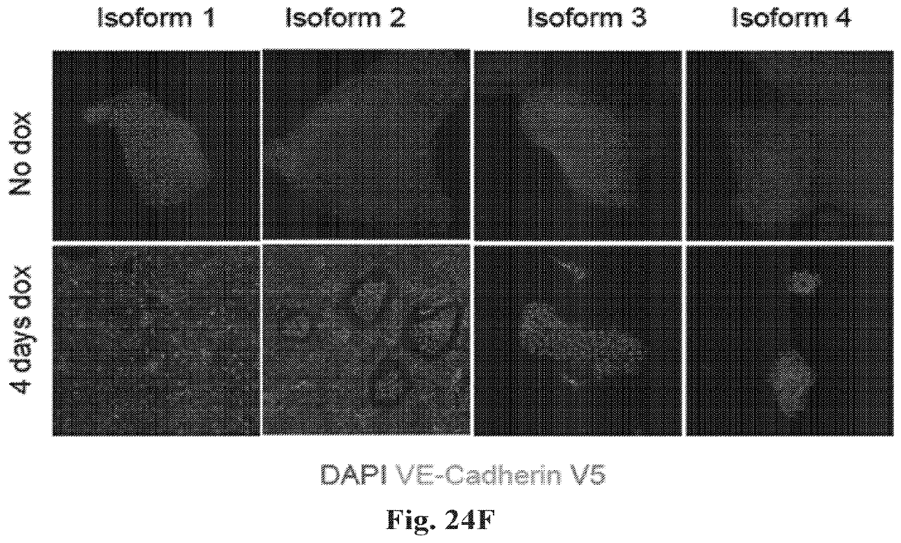

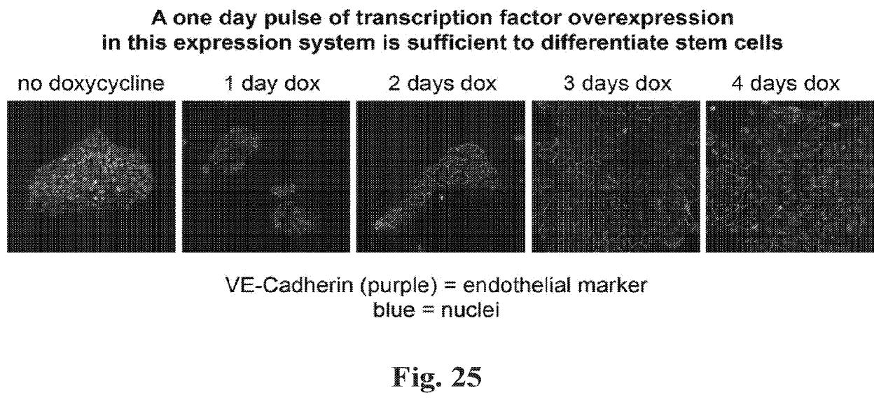

We have now validated 10 TFs identified from the TFome loss-of-pluripotency screen and have been working to characterize the cell types generated by them. Whereas the initial TFome screens used a lentiviral expression library, for validation and follow-on characterization we have found it preferable to use a piggyBac transposon system as this streamlines the validation pipeline and also offers the ability to optimize TF-directed hiPSC differentiation by adjusting the relative amounts of the piggyBac TF construct and the Super-piggyBac transposase vector. In general, validation and characterization experiments are conducted with PGP1 hiPSC bearing the integrated TFome construct in pluripotency reinforcing media that have been induced with dox for 4 days, at which point (depending on the particular experiment) we re-assess efficiency of differentiation by measuring loss of expression of pluripotency genes NANOG, SOX2 or OCT4/POU5F1, stain with a variety of cell type markers, and analyze RNA expression profiles obtained by RNA-seq. Additional characterizations of the cell types generated by the TF may use other media, conditions, or endpoints (as exemplified immediately below). We illustrate results from a few of these validation and characterization experiments in FIG. 24A-24E, but also report that surprising stories are beginning to emerge for some of these TFs. For instance, despite its sequence homology to neuron-inducing factors NEUROG1 and NEUROG2 (from which it derives its name), NEUROG3 is known in the literature as a pancreatic factor and has not, to our knowledge, been associated with neuron induction. Nevertheless, we find that NEUROG3 is a potent neuron-inducing factor when over-expressed alone in hiPSCs. Gene ontology analysis of RNA-seq data indicated strongly that it is involved in neuron differentiation, and cells generated by NEUROG3 expressed a panel of neuronal markers but not most pancreatic markers. Finally, we found that NEUROG3-generated neurons become electrically active and generate −1 Hz spontaneous action potentials within 21 days post induction. Additional experiments are under way to explore the hypothesis that NEUROG3 can direct hiPSC to differentiate into pancreatic cells, if it is not expressed alone but instead along with another TF.

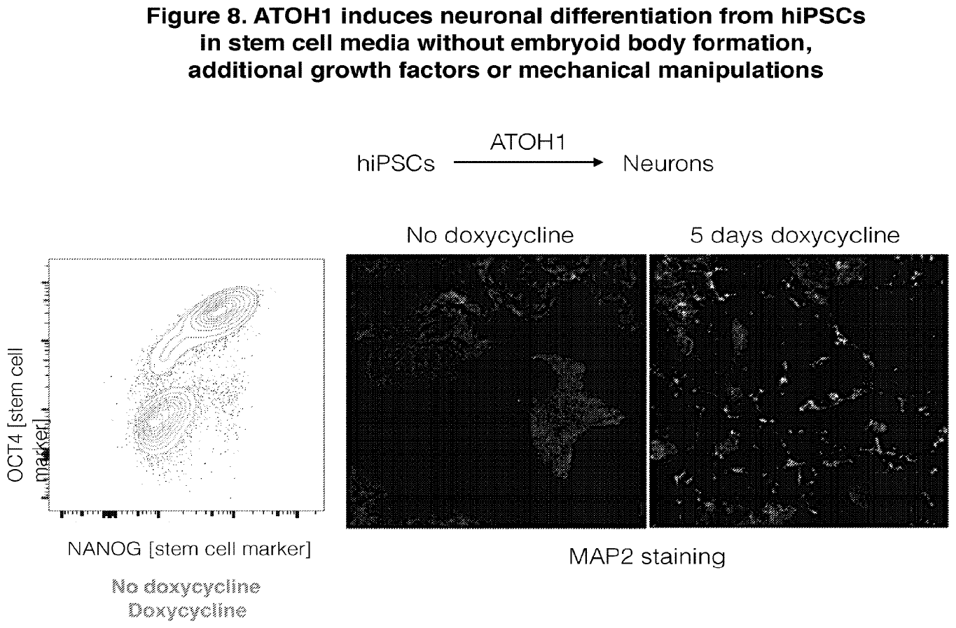

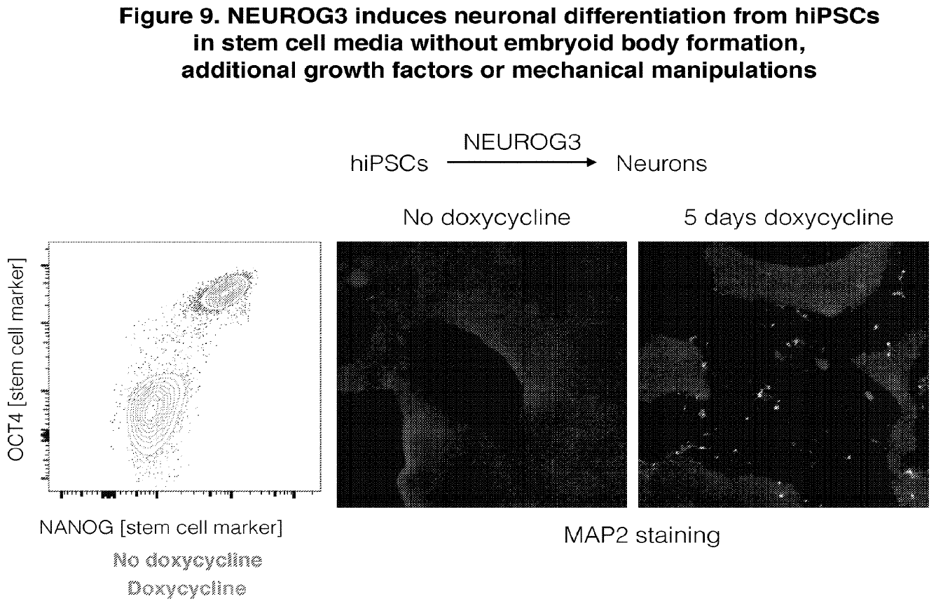

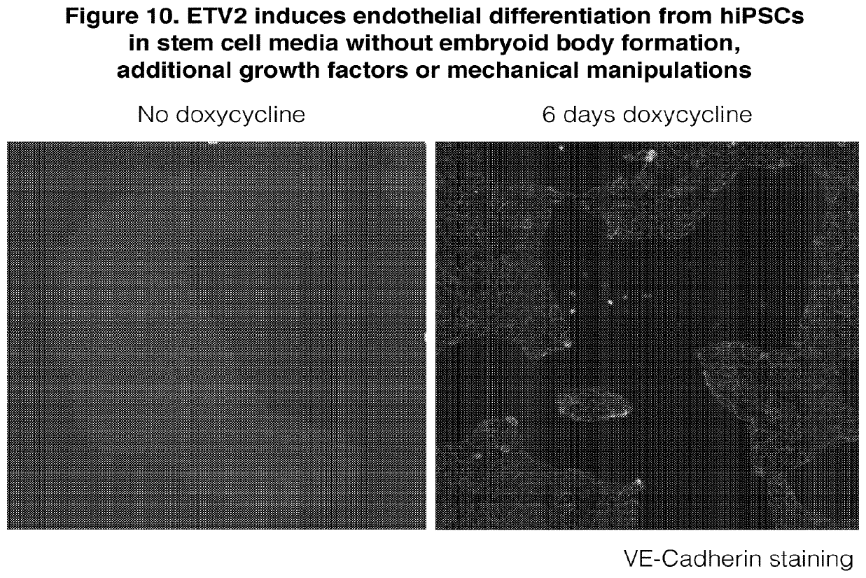

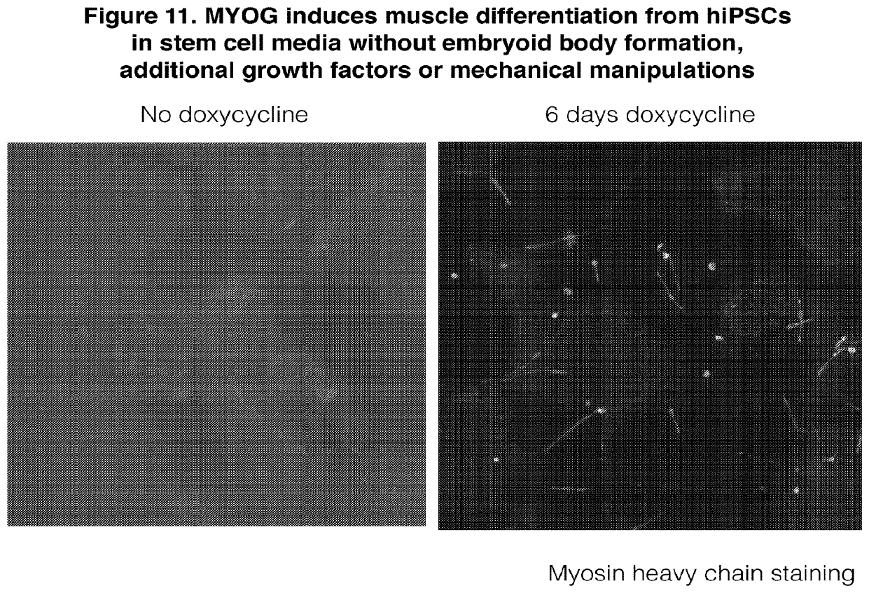

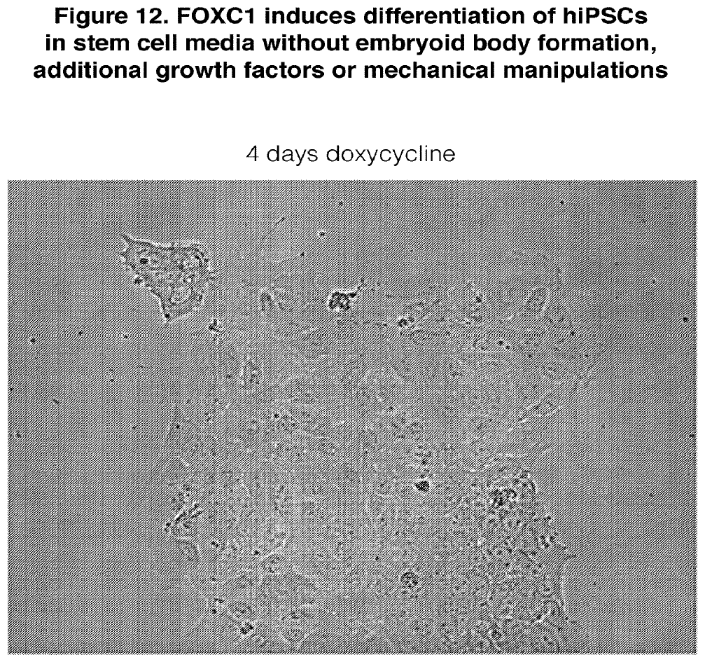

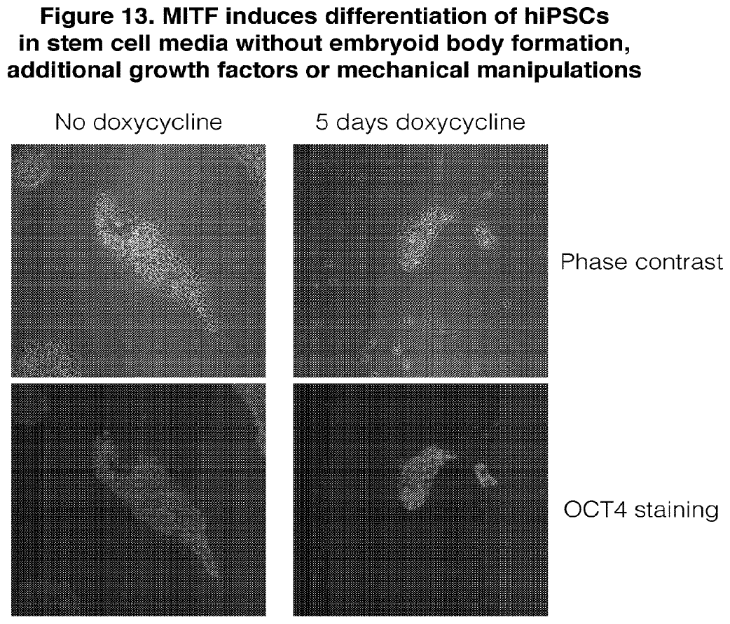

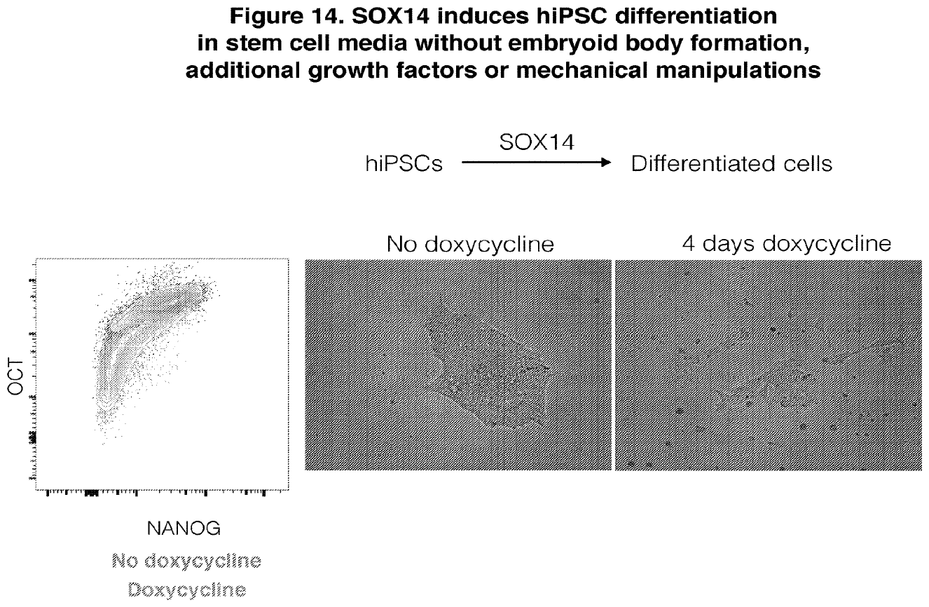

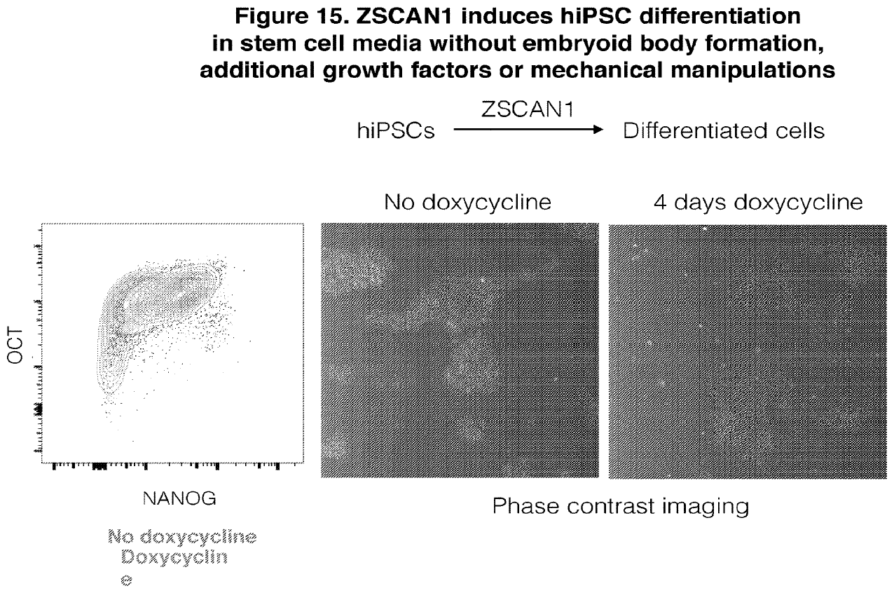

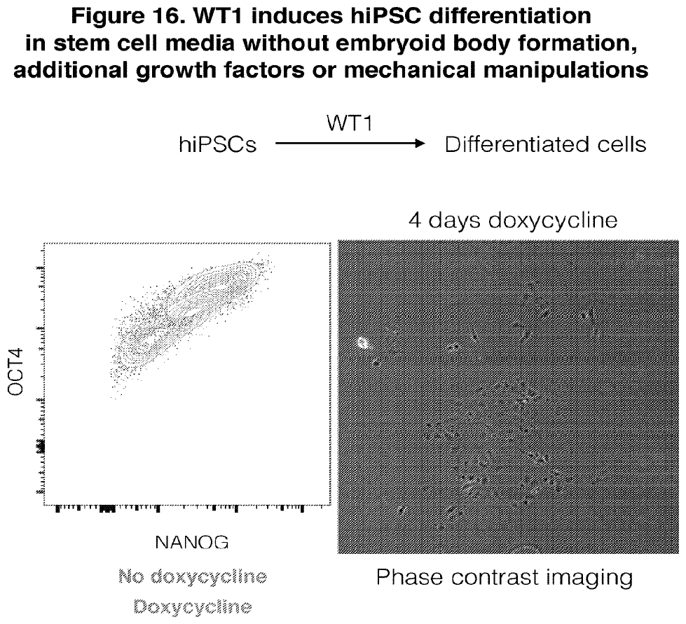

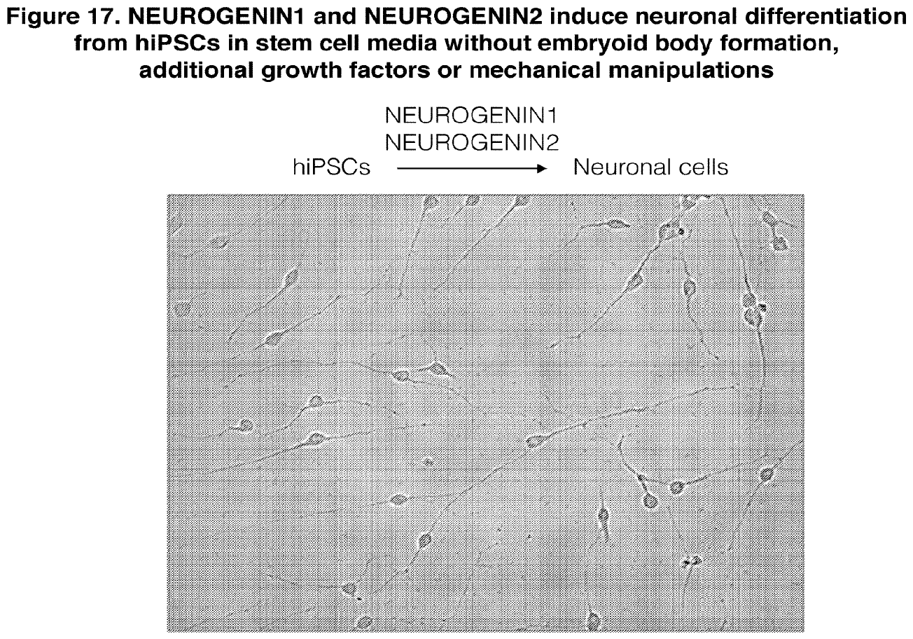

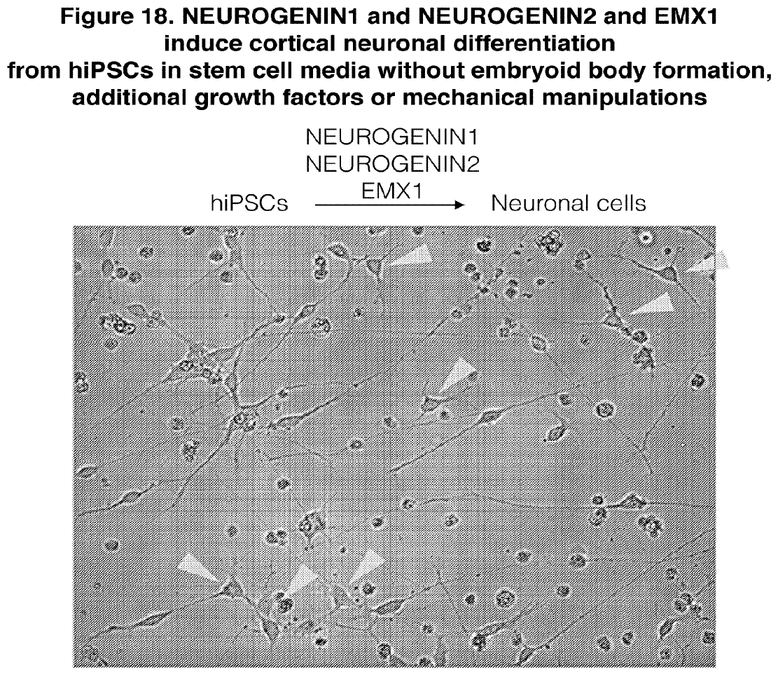

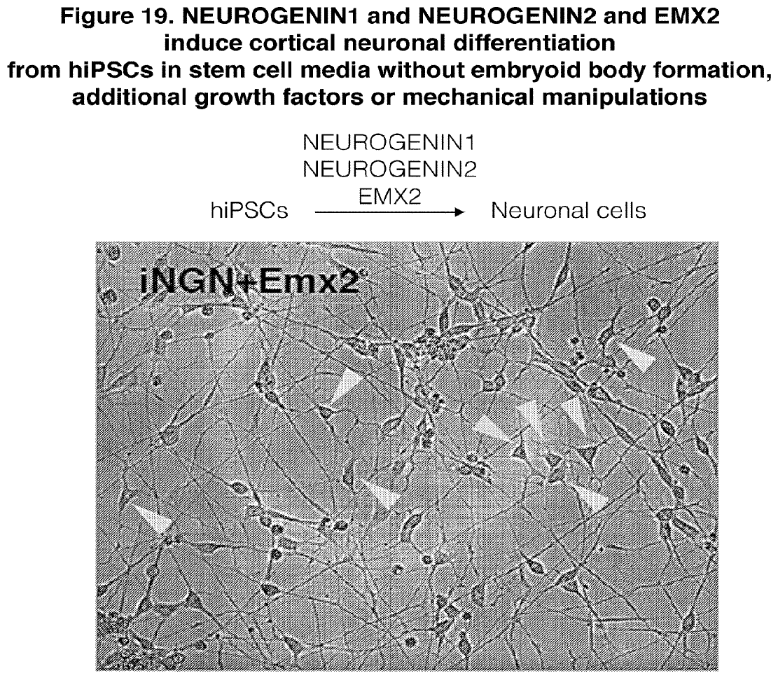

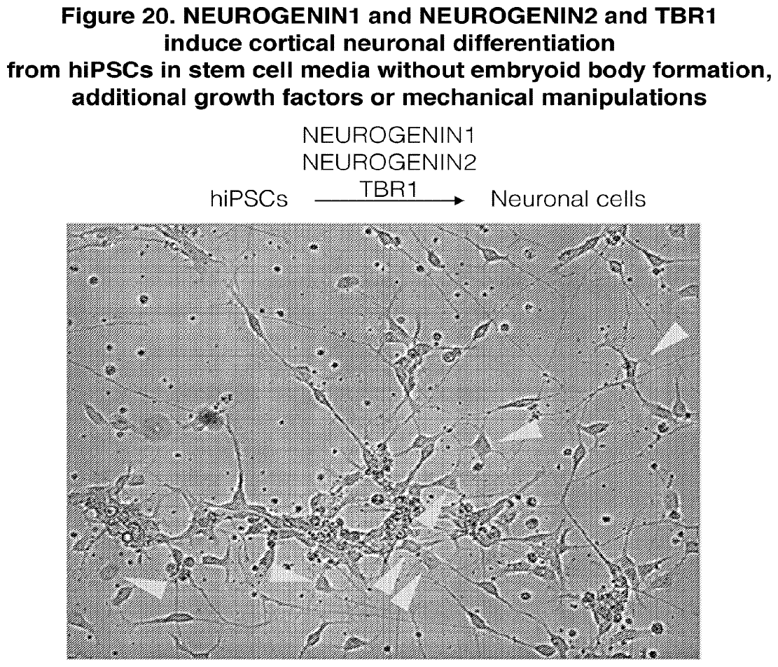

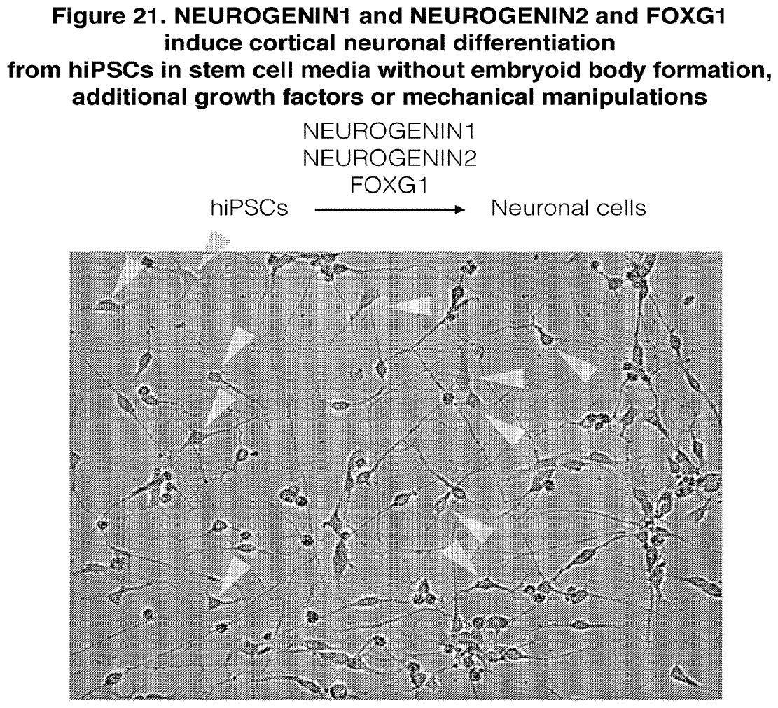

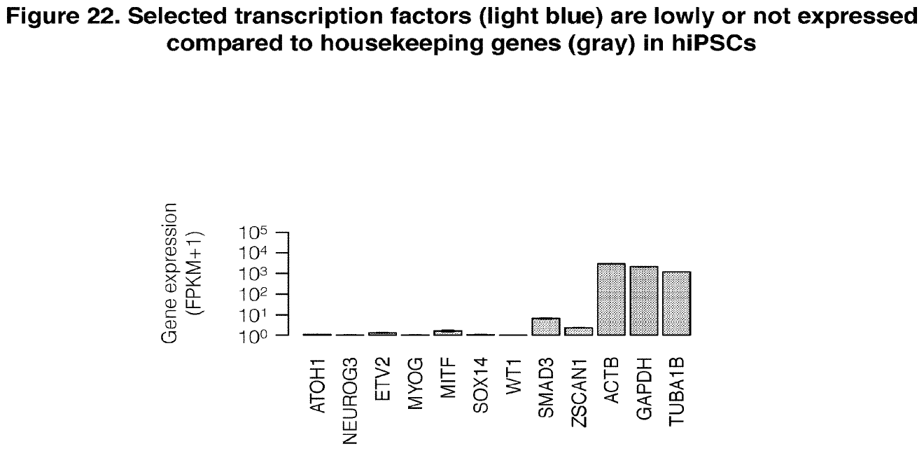

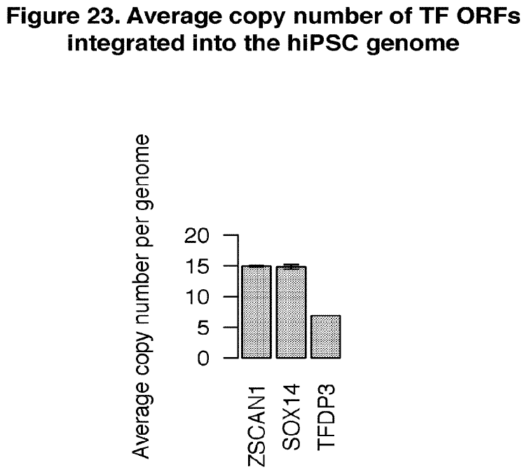

We have also begun to explore the differential effects of different TF isoforms on hiPSC differentiation. In an initial experiment, we tested all four known isoforms of the TF ETV2, which, as noted above, we had previously identified as directing differentiation towards an endothelial lineage. Our results indicated that under otherwise identical conditions, one isoform induces differentiation to endothelial cells with 90% efficiency while the others induce differentiation at levels of ˜50%, ˜20%, and 0% (see FIG. 8.E-F). We estimate that over 300 TFs have alternative gene isoforms, many of which may similarly have distinct efficiencies and effects on hiPSC differentiation, and we are preparing to collaborate with Marc Vidal's CCSB CEGS to generate a human TFome “2.0” that more systematically covers TF isoforms. We note that the ability to control isoform expression is a distinct advantage of expressing TFs through a library of expression constructs vs. Cas9 activation. While Cas9 may enable genes to be upregulated at their native promoters or enhancers, it offers no direct control over what isoforms are expressed.

Aside from validating and characterizing TFs identified by our initial TFome loss-of-pluripotency screens, we have also engaged in more focused testing and screening of combinations of TFome constructs for generating particular cell types, and we have also begun to explore the other end of the TFome spectrum—i.e., TFs that appeared in the screens to reinforce pluripotency rather than drive differentiation.

### Example 7

Pulsed Induction of Transcription Factor to Induce Overexpression and Differentiation

Methods: Human induced pluripotent stem cells were electroporated with the transcription factor in a transposon vector at high copy number. Doxycycline, which induces expression of the transcription factor, was added for varying number of days (one to four, or not added as a negative control). After 4 days in culture, cells were fixed with paraformaldehyde and stained with antibodies against VE-Cadherin, a marker for endothelial differentiation.

Results: Cells that received as little as one day of doxycycline successfully differentiated into endothelial cells with high efficiency, as indicated by strong VE-Cadherin staining at the cell membrane. The intensity of the staining was similar to cells induced for four days. Cells that did not receive doxycycline did not differentiate, as indicated by low VE-Cadherin staining, and strong nuclei staining for cell division.

### Example 8

NKX3-2 Induces Deterministic Stromal Cell Differentiation

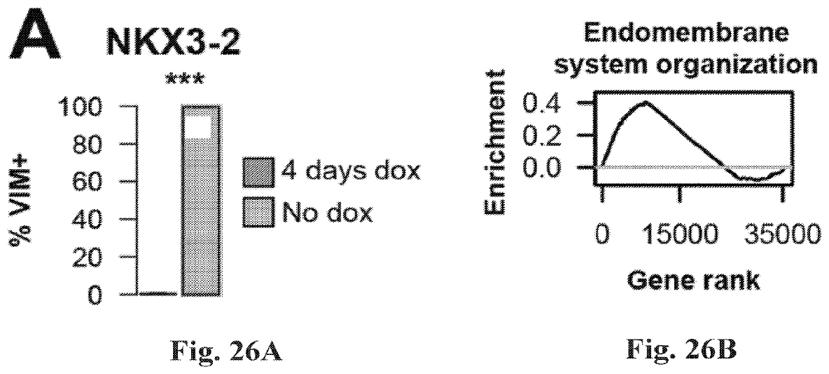

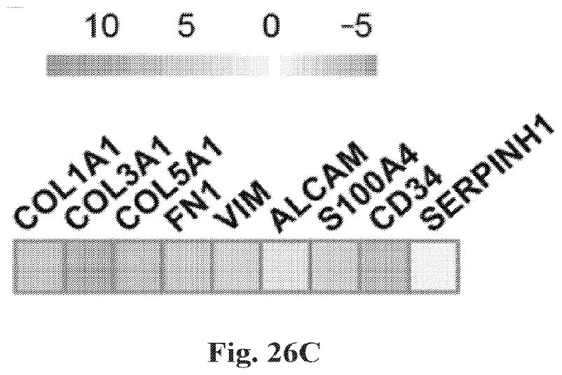

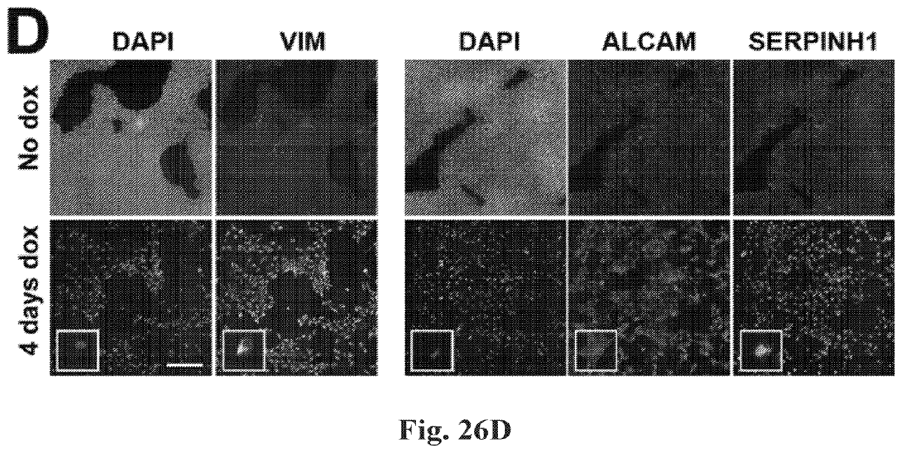

Methods: Human induced pluripotent stem cells were subjected to electroporation with ORF DNA of the transcription factor NKX3-2. Doxycycline was added to induce expression of the transcription factor for four days, then cells were dissociated, fixed, and stained for the stromal marker VIM. The percentage of VIM-positive cells was determined by flow cytometry (FIG. 26A). In another experiment, cells were differentiated for four days, and RNA was harvested from differentiated cells and stem cells, and then sequenced and quantified (FIG. 26B). The gene expression signature was used for gene ontology enrichment analysis. Additional stromal cell markers in the doxycycline-induced cells were evaluated by comparing them to stem cells on a log 2(stromal cell/stem cell) scale (FIG. 26C). Protein levels were determined by antibody-staining. A single cell is magnified in the insert (FIG. 26D).

Results: NKX3-2 overexpression in human stem cells potently induced stromal cell differentiation. This was confirmed by >99% of cells expressing the stromal cell marker VIM (FIG. 26 A), by the top gene ontology “endomembrane system organization” which is a signature of stromal cell secretion of extracellular matrix proteins (ECM) (FIG. 26B) and by gene expression of additional stromal cell markers such as collagen (COL1A1, COL3A1, COL5A1), fibronectin (FN1), stromal markers (ALCAM, S100A4), cell surface stromal markers (CD34), and collagen chaperone protein (SERPINH1) (FIG. 26C). Furthermore, induced strommal cells expressed high levels of these markers at the protein level (FIG. 26D).

Stromal markers were verified at the protein level by immunostaining, which indicated that expression of NKX3-2 alone induces stromal cells. A hallmark of stromal cells is to remodel the ECM, which can be assessed by the contraction of collagen. Indeed, NKX3-2-induced cells caused contraction when embedded within a collagen gel within 24 hours (FIG. 28A-28B), signifying functional stromal cells.

### Example 9

Experimental Methods (Used in Examples 1-5)

Annotation and Manual Curation of the TFome

Canonical human transcription factors were previously annotated by Vaquerizas et al. 2009) based on a computation search for genes that bind DNA in a sequence-specific manner, but are not enzymatic and do not form part of the core initiation complex, resulting in 1,591 TFs (classified as “a” and “b”, which have experimental evidence for regulatory function, and “other” as probable TFs with undefined DNA-binding domains; class “x” was excluded as having promiscuous DNA-binding domains). We further included TFs that were predicted but did not have experimental evidence (class “c”). The following major TF families were filled in according to HUGO Gene Nomenclature Committee (HGNC): zinc finger (includes C2H2-containing domains), homeodomain (includes LIM, POU, TALE, HOXL, NKL, PRD sub-families), basic helix-loop-helix and forkhead. Pseudogenes as annotated by HGNC were removed. Duplicated and unmapped genes were removed, and all genes were converted to approved gene names using the HGNC multi-symbol checker. The final target set of TFs in the human TFome contains 1,578 genes.

Assembly and Quality Control of the Human TFome

All TFs are cloned in pDONR-series standardized Gateway-compatible vector, building on Yang et al. 2011, Jolma et al., transOMIC technologies cDNAs, ORFs (transOMIC Inc), DNA Repository at Arizona State University, and codon-optimized synthesis by Gen 9.

Pooled Cloning of the Human TFome into pLIX403 Viral Vector

To perform pxooled LR cloning into the pLIX403 viral expression vector (Addgene 41395), each 96-well plate of DNA was combined into its own sub-pool and quantified using Qubit dsDNA Broad Range Assay Kit (Invitrogen Q32853). 75 ng of pENTR-TFome subpool was used for a LR Clonase II (Invitrogen 11791100) reaction overnight and digested with Proteinase K. 1 μL of the reaction was transformed into One Shot Stbl3 chemically competent cells (Invitrogen C737303), plated onto a 50 cm² LB Agar plate with 100 μg/mL Carbenicillin and incubated overnight at 30° C. Serial dilutions were performed to estimate the number colonies per plate. Approximately 10.000 to 20,000 colonies grew per plate, representing 100 to 200-fold coverage per 96-member sub-pool.

Colonies from each sub-pool plate were scraped and DNA was extracted using QIAGEN Plasmid Plus Midi Kit (QIAGEN 12943) to generate pLIX403-TFome subpools. To cloning efficiency and library coverage, pENTR and pLIX403 subpools were pooled to generate pENTR-TFome and pLIX403-TFome. 5 μg of each pool was sheared to 200 bp on a Covaris S2, and 1 μg was used for library preparation using NEBNext Ultra DNA Library Prep Kit for Illumina (NEB E7370L).

Lentiviral Production and Transduction

Lentiviruses were produced as before using 45 μg DNA for two 15 cm dishes, hiPSCs were transduced with TFome lentiviruses at a multiplicity of infection (MOI)=0.1.

Flow Cytometry Analysis and Fluorescence Activated Cell Sorting (FACS)

Cells were trypsinized, washed and resuspended in FACS buffer (PBS with 10% FBS). For surface antigens, live cells were stained with fluorophore-conjugated antibodies and viability dye CellTrace Calcein Blue, AM (Life Technologies. C34853) at 1×10⁷ cells/mL for 30 minutes on ice in the dark. For intracellular staining, cells were fixed using BD Cytofix fixation buffer (BD Biosciences, 554655) at 1×10⁷ cells/mL for 20 minutes, washed with BD Perm/Wash buffer (BD Biosciences, 554723), and permeabilized in BD Perm/Wash buffer for 10 minutes, then stained with antibodies and DAPI in the dark for 30 minutes. Stained cells were washed twice with FACS buffer, filtered into a strainer-capped tube (Falcon, 352235) and run on a BD LSRFortessa. Compensation for spectral overlap was determined by staining AbC Total Antibody Compensation Beads (Life Technologies. A10497) with single fluorophore-conjugated antibodies.

Transcriptome Library Preparation and Sequencing

TRIzol (Life Technologies. 15596-018) was added directly to cells and incubated for 3 minutes and used for RNA extraction using Direct-zol RNA MiniPrep (Zymo Research, R2050). RNA was quantified by Qubit RNA HS Kit (Molecular Probes, Q32852). 1 μg was used for Poly(A) isolation using Poly(T) beads (Bioo Scientific, 512979) and used for RNA-seq library preparation with unique molecular identifiers (UMIs) using NEXTflex Rapid Directional qRNA-seq kit (Bioo Scientific, 5130-02D). Libraries were amplified on a LightCycler real-time quantitative PCR machine (Roche) by spiking in SYBR Gold (Life Technologies, S11494). Mid-logarithm amplified libraries were collected and purified using AMPure XP beads (Agencourt, A63881). Libraries were quality controlled by TapeStation (Agilent) and quantitative PCR (KAPA Biosystems).

Transduction and Generation of Stable Cell Lines

For individual PiggyBac transductions, cells 500,000 to 800,000 cells were nucleofected using Nucleofector P3 solution (Lonza, V4XP-3032) using the Nucleofector X-Unit. Puromycin-resistant cells were selected and expanded.

Collagen Contraction Assay.

Collagen contraction assays were performed according to (52). NKX3-2 cells, grown in mTeSR1 either with or without doxycycline for 4 days, were dissociated and counted. 400,000 cells in 300 μl mTeSR1 were mixed with 150 μl collagen type I diluted to 3 mg/ml (BD, 354236) and 2 μl IM NaOH, and set in a 12-well plate used as a mold. Collagen gels were left to solidify at room temperature for 20 minutes, then 500 μl mTeSR1 was slowly added to the gels. The gel was dissociated from the mold by running a P200 tip along the edge of the well. The plate was incubated overnight and images were captured using a Zeiss Axio Zoom.V16 Stereo Zoom Microscope with a color AxioCam MRm camera and a PlanNeoFluar Z 1×/0.25 objective. The area of the gel was quantified in Fiji (53).

We claim:

1. A method of inducing differentiation of induced pluripotent stem cells, comprising:
delivering to the induced pluripotent stem cells a nucleic acid comprising an open reading frame encoding a transcription factor, the transcription factor protein, or an activator of transcription of the open reading frame encoding the transcription factor, whereby the amount of the transcription factor in the induced pluripotent stem cells is increased, and the induced pluripotent stem cells differentiate to form differentiated cells, wherein the transcription factor is selected from the group consisting of ASCL1; ASCL4; ATF1; ATF4; ATF7; ATOH1; ATTB1; ATXN7; BARHL2; BARX1; BATF3; BHLHA15; BOLA1; BOLA2; BOLA2B; BSX; CAMTA2; CDX1; CDX2; CEBPZ; CIZ1; CREB1; CREB3; CREB3L1; CREB3L4; CREBL2; DACH2; DLX1; DLX3; DMRT1; DRGX; DUXA; E2F2; EBF3; ELP3; EMX1; EN1; EN2; EPAS1; ETV1; ETV2; FIGLA; FLI1; FLJ12895; FOXB1; FOXC1; FOXD1; FOXD2; FOXD4L2; FOXE1; FOXF1; FOXF2; FOXH1; FOXI2; FOXL1; FOXN2; FOXO6; FOXP1; FOXR1; FOXR2; GBX2; GCM2; GLI1; GLIS1; GLIS3; GRHL1; GRHL2; GRHL3; GRLF1; GSC2; GZF1; HAND2; HES2; HES3; HES7; HIC2; HLX; HMGA1; HMX2; HOXA1; HOXA10; HOXA6; HOXB13; HOXB7; HOXB8; HOXB9; HOXC10; HOXC5; HOXD3; HSF1; HSFY1; ID3; ID4; IKZF1; IKZF3; INSM2; IRF2; IRF3; IRF7; IRF9; ISL2; KDM2A; KDM4E; KIAA0961; KLF6; KLF8; LBX2; LEUTX; LHX5; LMX1A; LOC51058; LOC91661; LYL1; MAEL; MAFK; MBNL3; MEF2B; MEF2C; MEIS1; MEIS3; MITF; MKX; MSC; MSGN1; MSX1; MXD3; MXD4; NEUROD4; NEUROD6; NEUROG1; NEUROG2; NEUROG3; NFIC; NFX1; NFYB; NKX2-2; NKX2-6; NKX2-8; NKX3-1; NKX3-2; NKX6-1; NKX6-3; NOTO; NPAS4; NR1B2; NR1H3; NR1H4; NR1I3; NR2F2; NR3C1; NR3C2; NRF1; NRL; OSR2; OTEX; OTP; OVOL1; OVOL2; PAX5; PAX9; PDX1; PEPP-2; PITX2; PKNOX2; PLAG1; PLAGL1; POU2F3; POU5F1B; PRDM5; PRDM6; PRDM7; PRDM9; PROX2; PRRX1; RAX2; REL; RELA; RFX1; RFX8; RFXANK; RUNX1; SALL3; SEBOX; SIM2; SIX2; SIX3; SIX4; SKIL; SMAD6; SMYD2; SOX100; SOX12; SOX13; SOX14; SOX21; SOX3; SPIB; SREBF1; TBX10; TBX18; TBX3; TBX5; TBX6; TCF1; TCF12; TCF15; TCF2; TCF21; TCF7; TCF7L2; TERF1; TFAP2B; TFCP2L1; TFDP2; TFDP3; TFE3; TGIF1; TLX1; TLX2; TLX3; TSC22D3; TUB; UBP1; UNCX; UNKL; USF1; VSX2; WDHD1; XBP1; YY1; ZBTB1; ZBTB2; ZBTB24; ZBTB25; ZBTB41; ZBTB44; ZBTB7C; ZBTB8B; ZFP36L2; ZFP64; ZFP92; ZFPM1; ZFX; ZGLP1; ZIC3; ZMAT1; ZMAT3; ZNF124; ZNF14; ZNF143; ZNF148; ZNF157; ZNF16; ZNF169; ZNF177; ZNF182; ZNF19; ZNF197; ZNF226; ZNF230; ZNF235; ZNF239; ZNF254; ZNF26; ZNF271; ZNF273; ZNF3; ZNF305; ZNF316; ZNF319; ZNF320; ZNF324; ZNF324B; ZNF326; ZNF333; ZNF34; ZNF347; ZNF350; ZNF395; ZNF396; ZNF404; ZNF408; ZNF415; ZNF426; ZNF44; ZNF440; ZNF441; ZNF442; ZNF443; ZNF449; ZNF45; ZNF454; ZNF470; ZNF474; ZNF480; ZNF490; ZNF499; ZNF506; ZNF507; ZNF512; ZNF514; ZNF516; ZNF518B; ZNF519; ZNF524; ZNF543; ZNF547; ZNF548; ZNF556; ZNF560; ZNF576; ZNF582; ZNF584; ZNF585B; ZNF592; ZNF593; ZNF594; ZNF599; ZNF600; ZNF616; ZNF626; ZNF639; ZNF643; ZNF646; ZNF652; ZNF653; ZNF66; ZNF660; ZNF664; ZNF668; ZNF671; ZNF678; ZNF682; ZNF683; ZNF692; ZNF706; ZNF709; ZNF714; ZNF716; ZNF717; ZNF718; ZNF720; ZNF721; ZNF729; ZNF749; ZNF75A; ZNF776; ZNF777; ZNF780B; ZNF783; ZNF785; ZNF791; ZNF799; ZNF8; ZNF808; ZNF835; ZNF84; ZNF841; ZNF843; ZNF85; ZNF852; ZSCAN22; ZSCAN23; ZSCAN29; ZSCAN5A; ZSCAN5C; ZXDB; MITF; NKX3-2; ZNF643; CDX2; ZNF777; SOX14; ZNF3; MKX; ZNF474; NEUROG1; DMRT1; PRDM5; NEUROG3; ZNF273; FOXP1; MXD4; NRL; E2F2; ZNF44; ZNF616; SOX12; HOXA10; GLI1; LMX1A; TBX10; EPAS1; HOXB7; PRDM7; RELA; OVOL2; BARX1; ATOH1; ZNF169; TSC22D3; NEUROG2; FOXD2; FOXI2; NR1B2; MEF2C; CAMTA2; HOXC5; ZNF230; IRF2; TFDP3; RUNX1; MSC; ZNF320; EN1; ZNF84; RAX2; NR1H3; DLX3; ZNF148; LHX5; BOLA2; TGIF1; BOLA1; ATF7; ID3; HMGA1; ZNF783; MAFK; GRHL1; FLI1; HES7; MAEL; ETV2; ZSCAN5A; HOXA1; FOXF2; KLF6; MBNL3; HES2; ATTB1; GRLF1; ZNF593; XBP1; ZNF326; MSGN1; BATF3; ID4; ZIC3; ZNF718; MXD3; ZNF426; ZNF706; ZNF652; FOXD1; GBX2; NEUROD4; ZNF490; ZNF85; SOX3; ZNF653; SIM2; HOXC10; ZNF26; FOXB1; ZNF668; ZNF576; NKX6-1; CDX1; MEF2B; DLX1; ZNF776; ZNF16; EN2; HOXA6; NKX2-8; CREBL2; DRGX; DUXA; ZNF396; ZNF692; VSX2; NR3C2; NR1I3; FIGLA; TLX1; HMX2; ASCL4; ZNF324B; ZNF75A; TCF21; BHLHA15; NKX6-3; ZNF720; TCF15; PITX2; HLX; ZNF124; SIX2; IKZF3; ZNF626; RFXANK; ATF4; ZNF678; ZNF514; GRHL2; FOXR2; ZNF660; CREB1; TFAP2B; ZMAT3; TCF7L2; FOXR1; OVOL1; HOXB13; HAND2; TCF12; UNKL; ZNF324; ZNF333; ZNF843; CREB3; ZBTB24; TERF1; ZNF785; FOXE1; OSR2; ZNF45; FOXL1; TBX3; ZNF157; NKX2-2; ZNF671; PAX9; NFIC; TBX6; NKX3-1; ZNF319; ZNF408; ZNF584; PKNOX2; SOX13; ZFP36L2; FLJ12895; NFX1; NR3C1; ZNF395; GLIS1; GLIS3; ZNF560; ZNF683; PLAGL1; SKIL; TCF1; UBP1; KLF8; ZNF239; TBX5; FOXF1; ZNF664; PRRX1; ISL2; FOXD4L2; ZNF143; KDM4E; ELP3; ZNF440; PEPP-2; PLAG1; TFE3; ZNF226; NEUROD6; TCF2; SMYD2; ZNF499; ZNF639; ZNF480; ZNF182; KDM2A; NPAS4; LOC51058; ATF1; SPIB; TLX2; ZBTB1; POU2F3; ZSCAN23; TUB; ZNF547; SIX4; KIAA0961; TFCP2L1; TCF7; ZNF177; YY1; ZNF585B; ZNF271; ZNF305; ZBTB2; OTP; NFYB; OTEX; ZNF443; ZNF852; ZNF646; ZNF835; ZNF682; ZNF66; ZNF235; SIX3; MEIS1; ZFPM1; ZNF441; LYL1; HOXB9; GSC2; ZBTB7C; ZNF518B; FOXH1; ZNF516; ZNF594; ZNF716; ZNF714; ZNF780B; ZNF470; CIZ1; RFX1; ZNF592; SOX21; LBX2; ZNF709; MSX1; LOC91661; PROX2; ZNF316; ZNF717; ZNF197; POU5F1B; ZBTB44; ZSCAN22; ZNF791; CREB3L1; GRHL3; REL; SOX10; SALL3; HSFY1; ZNF350; ZNF8; ZMAT1; NRF1; ZNF582; TBX18; ZFX; ZNF404; ZNF449; ZNF721; PDX1; SREBF1; GCM2; ZNF454; ZNF507; NR1H4; LEUTX; ZNF841; ZNF14; HES3; ASCL1; ZGLP1; INSM2; EMX1; HSF1; TFDP2; ZNF548; ZFP92; CREB3L4; CEBPZ; IKZF1; ZSCAN5C; FOXN2; ZNF524; ZFP64; EBF3; ZNF34; ZNF254; ZNF512; ZNF729; ZNF600; BOLA2B; BSX; WDHD1; ZNF19; ZNF543; PRDM9; ZXDB; ZBTB25; GZF1; NOTO; HOXD3; ZBTB41; ZNF442; IRF7; DACH2; IRF3; RFX8; NR2F2; ZBTB8B; PRDM6; ZNF808; ZNF556; ATXN7; ZSCAN29; NKX2-6; ZNF599; HOXB8; ZNF347; HIC2; BARHL2; ZNF506; FOXC1; ZNF415; PAX5; FOXO6; ZNF749; ZNF799; TLX3; UNCX; ETV1; SEBOX; MEIS3; IRF9; ZNF519; USF1; and SMAD6.

2. The method of claim 1 wherein a combination of two or more of said nucleic acids comprising an open reading frame encoding a transcription factor, two or more open reading frames encoding a transcription factor, two or more of said transcription factor proteins, or two or more of said activators of transcription of an open reading frame encoding a transcription factor, are delivered to the induced pluripotent stem cells.

3. The method of claim 1 further comprising:
contacting the differentiated cells with a test substance and observing a change in the differentiated cells induced by the test substance.

4. The method of claim 1 further comprising:
transplanting the differentiated cells into a patient.

5. The method of claim 4 wherein the induced pluripotent stem cells are derived from somatic cells of the patient.

6. The method of claim 4 further comprising:
contacting the differentiated cells with a test substance and observing a change in the differentiated cells induced by the test substance.

7. The method of claim 6 wherein the contacting is performed before the transplanting.

8. The method of claim 6 wherein the contacting is performed after the transplanting.

9. The method of claim 1 further comprising the step of sorting the differentiated cells using fluorescence activated cell sorting.

10. The method of claim 9 further comprising:
assaying the differentiated cells and determining a set of transcribed genes;
comparing the set of transcribed genes of the differentiated cells to one or more reference sets of transcribed genes from one or more reference tissues or cells; and
identifying a match between the differentiated cells and a reference tissue or cell.

11. The method of claim 9 further comprising:
assaying the differentiated cells and determining amounts of a set of transcribed genes;
comparing the amounts of the transcribed genes of the differentiated cells to one or more reference sets of amounts of transcribed genes from one or more reference tissues or cells; and
identifying a match between the differentiated cells and a reference tissue or cell.

12. The method of claim 9 further comprising the step of identifying differentiated cells as a type of differentiated cell by assaying morphological features of the differentiated cells and matching the morphological features to a reference tissue or cell's morphological features.

13. The method of claim 9 further comprising the step of identifying differentiated cells as a type of differentiated cell by assaying protein marker expression of the differentiated cells and matching the protein marker expression to a reference tissue or cell's protein marker expression.

14. The method of claim 9 further comprising the step of identifying differentiated cells as a type of differentiated cell by assaying a function and matching the function to a function of a reference tissue or cell.

15. The method of claim 9 wherein the differentiated cells are sorted on the basis of loss of expression of a stem cell marker.

16. The method of claim 15 wherein the stem cell marker is keratin sulfate cell surface antigen, TRA-1-60.

17. The method of claim 15 wherein the loss of expression of a stem cell marker is determined in a medium adapted for growth of stem cells.

18. An engineered human differentiated cell, comprising:
a nucleic acid comprising an open reading frame encoding a protein, wherein the protein is selected from the group consisting of ASCL1; ASCL4; ATF1; ATF4; ATF7; ATOH1; ATTB1; ATXN7; BARHL2; BARX1; BATF3; BHLHA15; BOLA; BOLA2; BOLA2B; BSX; CAMTA2; CDX1; CDX2; CEBPZ; CIZ1; CREB1; CREB3; CREB3L1; CREB3L4; CREBL2; DACH2; DLX1; DLX3; DMRT1; DRGX; DUXA; E2F2; EBF3; ELP3; EMX1; EN1; EN2; EPAS1; ETV1; ETV2; FIGLA; FLJ1; FU 12895; FOXB1; FOXC1; FOXD1; FOXD2; FOXD4L2; FOXE1; FOXF1; FOXF2; FOXH1; FOXI2; FOXL1; FOXN2; FOXO6; FOXP1; FOXR1; FOXR2; GBX2; GCM2; GLI1; GLIS1; GLIS3; GRHL1; GRHL2; GRHL3; GRLF1; GSC2; GZF1; HAND2; HES2; HES3; HES7; HIC2; HLX; HMGA1; HMX2; HOXA1; HOXA10; HOXA6; HOXB13; HOXB7; HOXB8; HOXB9; HOXC10; HOXC5; HOXD3; HSF1; HSFY1; ID3; ID4; IKZF1; IKZF3; INSM2; IRF2; IRF3; IRF7; IRF9; ISL2; KDM2A; KDM4E; KIAA0961; KLF6; KLF8; LBX2; LEUTX; LHX5; LMX1A; LOC51058; LOC91661; LYL1; MAEL; MAFK; MBNL3; MEF2B; MEF2C; MEIS1; MEIS3; MITF; MKX; MSC; MSGN1; MSX1; MXD3; MXD4; NEUROD4; NEUROD6; NEUROG1; NEUROG2; NEUROG3; NFIC; NFX1; NFYB; NKX2-2; NKX2-6; NKX2-8; NKX3-1; NKX3-2; NKX6-1; NKX6-3; NOTO; NPAS4; NR1B2; NR1H3; NR1H4; NR1I3; NR2F2; NR3C1; NR3C2; NRF1; NRL: OSR2; OTEX; OTP; OVOL1; OVOL2; PAX5; PAX9; PDX1; PEPP-2; PITX2; PKNOX2; PLAG1; PLAGL1; POU2F3; POU5F1B; PRDM5; PRDM6; PRDM7; PRDM9; PROX2; PRRX1; RAX2; REL; RELA; RFX1; RFX8; RFXANK; RUNX1; SALL3; SEBOX; SIM2; SIX2; SIX3; SIX4; SKIL; SMAD6; SMYD2; SOX10; SOX12; SOX13; SOX14; SOX21; SOX3; SPIB; SREBF1; TBX10; TBX18; TBX3; TBX5; TBX6; TCF1; TCF12; TCF15; TCF2; TCF21; TCF7; TCF7L2; TERF1; TFAP2B; TFCP2L1; TFDP2; TFDP3; TFE3; TGIF1; TLX1; TLX2; TLX3; TSC22D3; TUB; UBP1; UNCX; UNKL; USF1; VSX2; WDHD1; XBP1; YY1; ZBTB1; ZBTB2; ZBTB24; ZBTB25; ZBTB41; ZBTB44; ZBTB7C; ZBTB8B; ZFP36L2; ZFP64; ZFP92; ZFPM1; ZFX; ZGLP1; ZIC3; ZMAT1; ZMAT3; ZNF1124; ZNF14; ZNF143; ZNF148; ZNF157; ZNF16; ZNF169; ZNF177; ZNF182; ZNF19; ZNF197; ZNF226; ZNF230; ZNF235; ZNF239; ZNF254; ZNF26; ZNF271; ZNF273; ZNF3; ZNF305; ZNF316; ZNF319; ZNF320; ZNF324; ZNF324B; ZNF326; ZNF333; ZNF34; ZNF347; ZNF350; ZNF395; ZNF396; ZNF404; ZNF408; ZNF415; ZNF426; ZNF44; ZNF440; ZNF441; ZNF442; ZNF443; ZNF449; ZNF45; ZNF454; ZNF470; ZNF474; ZNF480; ZNF490; ZNF499; ZNF506; ZNF507; ZNF512; ZNF514; ZNF516; ZNF518B; ZNF519; ZNF524; ZNF543; ZNF547; ZNF548; ZNF556; ZNF560; ZNF576; ZNF582; ZNF584; ZNF585B; ZNF592; ZNF593; ZNF594; ZNF599; ZNF600; ZNF616; ZNF626; ZNF639; ZNF643; ZNF646; ZNF652; ZNF653; ZNF66; ZNF660; ZNF664; ZNF668; ZNF671; ZNF678; ZNF682; ZNF683; ZNF692; ZNF706; ZNF709; ZNF714; ZNF716; ZNF717; ZNF718; ZNF720; ZNF721; ZNF729; ZNF749; ZNF75A; ZNF776; ZNF777; ZNF780B; ZNF783; ZNF785; ZNF791; ZNF799; ZNF8; ZNF808; ZNF835; ZNF84; ZNF841; ZNF843; ZNF85; ZNF852; ZSCAN22; ZSCAN23; ZSCAN29; ZSCAN5A; ZSCAN5C; ZXDB; MITF; NKX3-2; ZNF643; CDX2; ZNF777; SOX14; ZNF3; MKX; ZNF474; NEUROG1; DMRT1; PRDM5; NEUROG3; ZNF273; FOXP1; MXD4; NRL; E2F2; ZNF44; ZNF616; SOX12; HOXA10; GLI1; LMX1A; TBX10; EPAS1; HOXB7; PRDM7; RELA; OVOL2; BARX1; ATOH1; ZNF169; TSC22D3; NEUROG2; FOXD2; FOXI2; NR1B2; MEF2C; CAMTA2; HOXC5; ZNF230; IRF2; TFDP3; RUNX1; MSC; ZNF320; EN1; ZNF84; RAX2; NR1H3; DLX3; ZNF148; LHX5; BOLA2; TGIF1; BOLA1; ATF7; ID3; HMGA1; ZNF783; MAFK; GRHL1; FLI1; HES7; MAEL; ETV2; ZSCAN5A; HOXA1; FOXF2; KLF6; MBNL3; HES2; ATTB1; GRLF1; ZNF593; XBP1; ZNF326; MSGN1; BATF3; ID4; ZIC3; ZNF718; MXD3; ZNF426; ZNF706; ZNF652; FOXD1; GBX2; NEUROD4; ZNF490; ZNF85; SOX3; ZNF653; SIM2; HOXC10; ZNF26; FOXB1; ZNF668; ZNF576; NKX6-1; CDX1; MEF2B; DLX1; ZNF776; ZNF16; EN2; HOXA6; NKX2-8; CREBL2; DRGX; DUXA; ZNF396; ZNF692; VSX2; NR3C2; NR1I3; FIGLA; TLX1; HMX2; ASCL4; ZNF324B; ZNF75A; TCF21; BHLHA15; NKX6-3; ZNF720; TCF15; PITX2; HLX; ZNF124; SIX2; IKZF3; ZNF626; RFXANK; ATF4; ZNF678; ZNF514; GRHL2; FOXR2; ZNF660; CREB1; TFAP2B; ZMAT3; TCF7L2; FOXR1; OVOL1; HOXB13; HAND2; TCF12; UNKL; ZNF324; ZNF333; ZNF843; CREB3; ZBTB24; TERF1; ZNF785; FOXE1; OSR2; ZNF45; FOXL1; TBX3; ZNF157; NKX2-2; ZNF671; PAX9; NFIC; TBX6; NKX3-1; ZNF319; ZNF408; ZNF584; PKNOX2; SOX113; ZFP36L2; FLJ12895; NFX1; NR3C1; ZNF395; GUIS1; GLIS3; ZNF560; ZNF683; PLAGL1; SKIL; TCF1; UBP1; KLF8; ZNF239; TBX5; FOXF1; ZNF664; PRRX1; ISL2; FOXD4L2; ZNF143; KDM4E; ELP3; ZNF440; PEPP-2; PLAG1; TFE3; ZNF226; NEUROD6; TCF2; SMYD2; ZNF499; ZNF639; ZNF480; ZNF182; KDM2A; NPAS4; LOC51058; ATF1; SPIB; TLX2; ZBTB1; POU2F3; ZSCAN23; TUB; ZNF547; SIX4; KIAA0961; TFCP2L1; TCF7; ZNF177; YY1; ZNF585B; ZNF271; ZNF305; ZBTB2; OTP; NFYB; OTEX; ZNF443; ZNF852; ZNF646; ZNF835; ZNF682; ZNF66; ZNF235; SIX3; MEIS1; ZFPM1; ZNF441; LYL1; HOXB9; GSC2; ZBTB7C; ZNF518B; FOXH1; ZNF516; ZNF594; ZNF716; ZNF714; ZNF780B; ZNF470; CIZ1; RFX1; ZNF592; SOX21; LBX2; ZNF709; MSX1; LOC91661; PROX2; ZNF316; ZNF717; ZNF197; POU5F1B; ZBTB44; ZSCAN22; ZNF791; CREB3L1; GRHL3; REL; SOX10; SALL3; HSFY1; ZNF350; ZNF8; ZMAT1; NRF1; ZNF582; TBX18; ZFX; ZNF404; ZNF449; ZNF721; PDX1; SREBF1; GCM2; ZNF454; ZNF507; NR1H4; LEUTX; ZNF841; ZNF14; HES3; ASCL1; ZGLP1; INSM2; EMX1; HSF1; TFDP2; ZNF548; ZFP92; CREB3L4; CEBPZ; IKZF1; ZSCAN5C; FOXN2; ZNF524; ZFP64; EBF3; ZNF34; ZNF254; ZNF512; ZNF729; ZNF600; BOLA2B; BSX; WDHD1; ZNF19; ZNF543; PRDM9; ZXDB; ZBTB25; GZF1; NOTO; HOXD3; ZBTB41; ZNF442; IRF7; DACH2; IRF3; RFX8; NR2F2; ZBTB8B; PRDM6; ZNF808; ZNF556; ATXN7; ZSCAN29; NKX2-6; ZNF599; HOXB8; ZNF347; HIC2; BARHL2; ZNF506; FOXC1; ZNF415; PAX5; FOXO6; ZNF749; ZNF799; TLX3; UNCX; ETV1; SEBOX; MEIS3; IRF9; ZNF519; USF1; and SMAD6, wherein the open reading frame is intronless.

19. The engineered human differentiated cell of claim 18 wherein the nucleic acid comprising an open reading frame is selected from the group consisting of a cDNA, a synthetic nucleic acid, and an mRNA.

20. The method of claim 1 wherein the nucleic acid comprising an open reading frame is selected from the group consisting of a cDNA, a synthetic nucleic acid, and an mRNA.

21. The method of claim 1 wherein the transcription factor is NKX3-2 and stromal cells are formed.

22. The method of claim 1 wherein the open reading frame is delivered, and is maintained in the induced pluripotent stem cells at a high copy number.

23. The method of claim 1 wherein the open reading frame is delivered, and is maintained in the induced pluripotent stem cells at a copy number of greater than 10 per cell.

24. The method of claim 1 wherein the activator of transcription is delivered and the delivery is for less than 5 days.

25. The method of claim 1 wherein the activator of transcription is delivered and the delivery is for less than 4 days.

26. The method of claim 1 wherein the activator of transcription is delivered and the delivery is for less than 3 days.

27. The method of claim 1 wherein the activator of transcription is delivered and the delivery is for less than 2 days.

28. An engineered human differentiated cell comprising a nucleic acid comprising an open reading frame that encodes transcription factor NKX3-2, wherein the open reading frame is intronless.

29. The engineered human differentiated cell of claim 28 wherein the cell is a stromal cell.

30. The engineered human differentiated cell of claim 28 wherein the cell is part of a three-dimensional tissue.

31. A method of inducing differentiation of induced pluripotent stem cells, comprising:
inducing increased expression of a NKX3-2 gene, whereby the induced pluripotent stem cells differentiate to form differentiated cells.

32. A method of inducing differentiation of induced pluripotent stem cells, comprising:
inducing expression of a NKX3-2, wherein the induced pluripotent stem cells differentiate to form differentiated cells.
